(19) United States
(12) Patent Application Publication
(10) Pub. No.: US 2025/0377366 A1
(43) Pub. Date: Dec. 11, 2025

(54) ASSESSMENT OF CHRONIC LIVER DISEASE

(71) Applicant: Roche Diagnostics Operations, Inc., Indianapolis, IN (US)

(72) Inventors: Konstantin Kroeniger, Barcelona (ES); Magdalena Swiatek-De Lange, Wroclaw (DE); David Morgenstern, Indianapolis, IN (US)

(21) Appl. No.: 19/308,547

(22) Filed: Aug. 25, 2025

Related U.S. Application Data
(63) Continuation of application No. PCT/EP2023/054608, filed on Feb. 23, 2023.

(51) Int. Cl. G01N 33/68; C12Q 1/48

(52) U.S. Cl. CPC G01N 33/6893; C12Q 1/48; G01N 2333/4745; G01N 2333/5421; G01N 2333/9108; G01N 2800/085

(57) ABSTRACT

The present invention relates to a method for assessing chronic liver disease in a subject, said method comprising (a) determining an amount of the biomarker Insulin-like growth factor-binding protein 3 (IGFBP3) in a sample from said subject; (b) determining an amount of the biomarker gamma-glutamyltransferase (GGT) in said sample; (c) comparing the amounts of the biomarkers determined in steps (a) and (b) to references for said biomarkers and/or calculating a score for assessing chronic liver disease; and (d) assessing chronic liver disease in said subject based on the comparison and/or the calculation made in step (c). The present invention further relates to computer-implemented methods, databases, devices, and uses related thereto.

The present invention relates to a method for assessing chronic liver disease in a subject, said method comprising (a) determining an amount of the biomarker Insulin-like growth factor-binding protein 3 (IGFBP3) in a sample from said subject; (b) determining an amount of the biomarker gamma-glutamyltransferase (GGT) in said sample; (c) comparing the amounts of the biomarkers determined in steps (a) and (b) to references for said biomarkers and/or calculating a score for assessing chronic liver disease; and (d) assessing chronic liver disease in said subject based on the comparison and/or the calculation made in step (c). The present invention further relates to computer-implemented methods, databases, devices, and uses related thereto.

Chronic liver disease (CLD) is a major cause of global mortality and morbidity. CLD, regardless of its cause, follows a common pathway by which the liver is repeatedly or continuously damaged, resulting in the formation of scar tissue, fibrosis. Patients with CLD are at an increased risk of developing liver fibrosis, cirrhosis and liver failure. In addition, they are at significant risk to develop primary liver cancer, in particular hepatocellular carcinoma (HCC). Liver fibrosis is initiated by a variety of factors causing death of hepatocytes, prominently viral infections (hepatitis B virus (HBV), hepatitis C virus (HCV)), alcohol, and diet (non-alcoholic fatty liver disease (NAFLD)). Those factors lead to activation of hepatic stellate cells (HSCs), which is the main mechanism leading to liver fibrosis (Tsukada et al, Clin Chim Acta 2006; 364:33-60). Symptoms and diagnostics of liver fibrosis are known in the art, e.g. from medical textbooks and from Bataller & Brenner (2005), J Clin Invest 115:209, and Guo & Lu (2020), J Clin transl Hepatol 8(3):304. Typically, liver fibrosis entails excessive accumulation of extracellular matrix proteins, including collagen, in the liver. There are several staging systems for staging liver fibrosis, e.g. the Ishak, METAVIR, and Batts-Ludwig scoring systems, reviewed e.g. in Chowdhury and Mehta (2022), Clin Exp Med, doi.org/10.1007/s10238-022-00799-z.

As the result of vaccination programs and therapy development, HBV and HCV driven CLD has been declining. However, non-alcoholic fatty liver disease (NAFLD) and nonalcoholic steatohepatitis (NASH) are becoming increasingly prevalent and represent a major risk for CLD (Estes et al. (2018), J Hepatol.69(4):896).

Selection of optimal diagnostics and management for a patient suffering from liver disease is essential and relies on accurate assessment and monitoring of the fibrosis stage. Liver biopsy is currently the gold standard to assess fibrosis. Liver biopsy has some relevant disadvantages and inconveniences, including sampling error caused by small sample size (Bravo et al., N Engl J Med 2001; 344:495-500), inter-observer variability between pathologists evaluating biopsies, and complications associated with percutaneous liver biopsy. Thus, in the last 20 years, the number of biopsies performed has declined sharply.

Instead, non-invasive diagnostic scores, biomarkers, and imaging modalities, such as Aspartate aminotransferase to platelet ratio index (ASTI), FIB-4, FibroTest, hyalorunan, etc., gained popularity, as they are cheaper, better tolerated, safer, and more acceptable to the patient than liver biopsy (Lurie et al., World J Gastroenterol 2015; 21(41):11567-11583). Currently, collagens and their fragments, forming the main component of fibrotic scars, are validated as biomarkers for the assessment of fibrosis. There are e.g. tests for amino-terminal propeptides of procollagen type III, PIIINP and PRO-C3 (Karsdal et al., Liver Int 2020; 40:736-750). Enhanced liver fibrosis (ELF™) test, comprising hyaluronic acid, PIIINP and TIMP-1, has a good sensitivity and specificity for severe liver fibrosis (Xie et al, PLOS One 2014,doi.org/10.1371/journal.pone.0092772). Insulin-like growth factor-binding protein 3 (IGFBP3) has been proposed as a fibrosis biomarker as a stand-alone marker or in relation to IGFI (Correa et al., World J Hepatol 2016; 8(17):739-748; Volzke et al., European Journal of Endocrinology 2009; 161(5): 705-713). Gamma-glutamyltransferase (GGT) activity is an established biomarker of liver function, however, not as popular as other liver function tests (LFT), such as bilirubin, albumin, alanine aminotransferase (ALT) and alkaline phosphatase (Dillon et al., Annals of Clinical Biochemistry 2016, Vol. 53(6) 629-631). GGT is a part of several diagnostic panels, e.g. ALFI, Fibrotest, or HepaScore. IL-8 is a strong predictor of increased fibrotic liver injury compared to established markers of hepatic fibrosis (Glass et al., Hepatol Commun. 2018;2:1344-1355).

However, none of the currently available biomarkers by itself has sufficient accuracy for diagnosing fibrosis, therefore they are usually combined to form predictive scores. Among the predictive scores fibrosis-4 index (FIB-4) index, NAFLD fibrosis score (NFS), the BARD score, FibroTest, HepatoScore, hepamet fibrosis score (HFS), and AST to platelets ratio index (APRI) score, are the most widely used. However, the European Association for the Study of the Liver (EASL) guidelines recommend that for the identification of advanced fibrosis or cirrhosis, serum biomarkers/scores and/or transient elastography (TE) are less accurate, and it is important to confirm these advanced stages by liver biopsy (European Association for the Study of the Liver (EASL); European Association for the Study of Diabetes (EASD); European Association for the Study of Obesity (EASO)). EASL-EASD-EASO Clinical Practice Guidelines for the management of non-alcoholic fatty liver disease. J Hepatol 2016; 64:1388-1402).

In the last two decades, transient elastography (TE) has emerged as a quantitative imaging approach to non-invasively assess liver fibrosis. Elastography may be performed with ultrasound (US) or magnetic resonance imaging (MRI). The underlying principle is that liver tissue stiffness and other tissue mechanical properties can be estimated quantitatively by analyzing the propagation of shear waves introduced into those tissues (Ophir et al., Ultrasound Imaging 1991; 13:111-134). However, TE is subject to several technical and patient-related limitations like frequent requirement for recalibration, a significant proportion of unreliable measurements, and a higher technical failure rate in the presence of confounders such as acute inflammation, narrow intercostal space, ascites, and obesity (Castera et al., Hepatology 2010;51:828-835).

Thus, although several non-invasive tools are available, they perform sub-optimally, leading to a large number of unnecessary invasive procedures, such as biopsies. Consequently, there is an urgent need for new biomarkers in CLD patients to support patient management and facilitate the evaluation of new drugs. It is therefore an objective of the present invention to provide improved means and methods for assessing chronic liver disease avoiding at least in part the drawbacks of the prior art.

This problem is solved by the means and methods of the present invention, with the features of the independent claims. Preferred embodiments, which might be realized in an isolated fashion or in any arbitrary combination are listed in the dependent claims.

In accordance, the present invention relates to a method for assessing chronic liver disease in a subject, said method comprising
(a) determining an amount of the biomarker Insulin-like growth factor-binding protein 3 (IGFBP3) in a sample from said subject;
(b) determining an amount of the biomarker glutamyltransferase (GGT) in said sample;
(c) comparing the amounts of the biomarkers determined in steps (a) and (b) to references for said biomarkers and/or calculating a score for assessing chronic liver disease; and
(d) assessing chronic liver disease in said subject based on the comparison and/or the calculation made in step (c).

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” in an embodiment refer to “comprising one or more”, i.e. are equivalent to “comprising at least one”. In accordance, expressions relating to one item of a plurality, unless otherwise indicated, in an embodiment relate to at least one such item, in a further embodiment a plurality thereof; thus, e.g. identifying “a cell” relates to identifying at least one cell, in an embodiment to identifying a multitude of cells.

In accordance, the term “at least one”, as used herein, means that one or more of the items referred to following the term may be used or be present. For example, if the term indicates that at least one sampling unit shall be used, this may be understood as one sampling unit or more than one sampling units, i.e. two, three, four, five or any other number. Depending on the item the term refers to, the skilled person understands as to what upper limit the term may refer, if any.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The methods specified herein below, in an embodiment, are in vitro methods. The method steps may, in principle, be performed in any arbitrary sequence deemed suitable by the skilled person, but in an embodiment are performed in the indicated sequence; also, one or more, in an embodiment all, of said steps may be assisted or performed by automated equipment. Moreover, the methods may comprise steps in addition to those explicitly mentioned above. Furthermore, the terms “first”, “second”, “third” and the like in the description and in the claims are used for distinguishing between similar elements and not necessarily for describing a sequential or chronological order.

As used herein, if not otherwise indicated, the term “about” relates to the indicated value with the commonly accepted technical precision in the relevant field, in an embodiment relates to the indicated value ±20%, in a further embodiment ±10%, in a further embodiment ±5%. Further, the term “essentially” indicates that deviations having influence on the indicated result or use are absent, i.e. potential deviations do not cause the indicated result to deviate by more than ±20%, in a further embodiment ±10%, in a further embodiment ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. In an embodiment, a composition consisting essentially of a set of components will comprise less than 5% by weight, in a further embodiment less than 3% by weight, in a further embodiment less than 1% by weight, in a further embodiment less than 0.1% by weight of non-specified component(s).

The term “assessing”, as used herein, refers to establishing information about the status of the indicated disease or condition, in particular its severity, symptoms, localization, prognosis and/or other relevant information. Said assessing, in an embodiment, is an aid in diagnosing the indicated disease; as the skilled person will understand, establishing a diagnosis may be based on the aforesaid assessment, however, in a further embodiment, is based on the aforesaid assessment in combination with further diagnostic information, such as anamnesis data, general physical, mental examination findings, and/or additional metabolic data. Thus, assessing chronic liver disease may relate to assessing whether a subject suffers from chronic liver disease, is at risk of suffering from chronic liver disease, exhibits a medical condition which deteriorates with respect to chronic liver disease, to establishing the disease stage of chronic liver disease of a subject, and/or establishing a prognosis with respect to chronic liver disease. Accordingly, assessing as used herein includes diagnosing chronic liver disease, predicting the risk for developing chronic liver disease, and/or predicting any deterioration of the health condition of the subject, in particular, with respect to signs and symptoms accompanying chronic liver disease. Assessment referred to herein may also be the assessment of a risk of developing chronic liver disease. In a further embodiment, assessment may be the prediction of the risk that the subject's (health) condition of the subject will deteriorate. Moreover, it will be understood that if the risk of developing chronic liver disease or risk of the deterioration of the health condition is predicted, typically, the prediction is made within a predictive window. More typically, said predictive window is of from 1 day to 6 months, in a further embodiment of from one week to 2 months.

Assessing as referred to herein may relate to a rule-in assessment, i.e. to identifying a subject as belonging to a group of subjects sharing a common feature, e.g. suffering from chronic liver disease. Assessing, however, may also relate to a rule-out assessment, i.e. to identifying a subject as not belonging to a group of subjects sharing a common feature, e.g. as not suffering from chronic liver disease. Thus, the assessment may aid in establishing a diagnosis; the assessment may, however, also aid in excluding a diagnosis. Thus, the method as specified may in particular be comprised in a method of monitoring chronic liver disease.

In view of the above, assessing chronic liver disease in particular may include or be aiding in diagnosis to assess the stage of liver fibrosis (hepatic fibrosis); in an embodiment to be applied in a specialist or tertiary care setting, in particular with access to a laboratory environment where automated immunoassays can be run; and/or an aid in the diagnosis and assessment of the severity of liver fibrosis in patients with signs and symptoms of chronic liver disease, in an embodiment in conjunction with other laboratory findings and clinical assessments. Also in an embodiment, assessing chronic liver disease in particular may include or be diagnosing liver fibrosis, in an embodiment diagnosing advanced liver fibrosis; staging chronic liver disease, staging liver fibrosis, and/or differentiating between non-advanced liver fibrosis and advanced liver fibrosis. Assessing chronic liver disease, may, however, also include or be aiding in excluding one or more specific stage(s) of liver fibrosis (hepatic fibrosis); and/or an aid in the exclusion of severe liver fibrosis in patients with signs and symptoms of chronic liver disease, in an embodiment in conjunction with other laboratory findings and clinical assessments. Also in an embodiment, assessing chronic liver disease in particular may include or be excluding liver fibrosis, in an embodiment excluding advanced liver fibrosis.

As will be understood by those skilled in the art, the assessment made in accordance with the present invention, although usually preferred to be, may not be correct for 100% of the investigated subjects. However, the term typically requires that a statistically significant portion of subjects can be correctly assessed. Whether a portion is statistically significant can be determined without further ado by the person skilled in the art using various well known statistic evaluation tools, e.g., determination of confidence intervals, p-value determination, Student's t-test, Mann-Whitney test, etc. Details may be found in Dowdy and Wearden, Statistics for Research, John Wiley & Sons, New York 1983. Typically envisaged confidence intervals are at least 50%, at least 60%, at least 70%, at least 80%, at least 90%, at least 95%. The p-values are, typically, 0.2, 0.1, 0.05.

The term “chronic liver disease”, as used herein, relates to a chronic disease that affects liver function. The chronic liver disease may be accompanied by steatosis (such as NASH) or, in an embodiment, not be accompanied by steatosis. Chronic liver disease is, in an embodiment, caused by viral infection, bacterial infection, parasite infection, drugs, chemical intoxication, fatty liver disease, autoimmune hepatitis, and/or environmental contamination. Viral infections that cause chronic liver disease are, in an embodiment, infection with HAV (hepatitis A virus), HBV (hepatitis B virus), HCV (hepatitis C virus), HDV (hepatitis D virus), HEV (hepatitis E virus), CMV (cytomegalovirus), and/or EBV (Epstein-Barr virus). Drugs or chemicals that cause chronic liver disease are well known in the art. Well-known drugs and/or chemicals are alcohol, in particular alcohol abuse, carbon-tetrachloride, amethopterin, tetracycline, acetaminophen, fenoprofen, cyclopeptides, monomethylhydrazine, sulphamethizole, urolucosil, sulphacetamide, and silver sulphadiazine. Accordingly, the method of the present invention allows for diagnosing chronic liver disease in subjects after contact with one of the aforesaid biological or chemical agents. The term “fatty liver disease” is well known in the art. In an embodiment, the term refers to an impairment of the liver caused by of a surplus of triacylglycerides that accumulate in the liver and form large vacuoles. Fatty liver disease may e.g. result from alcohol abuse, diabetes mellitus, nutritional defects and wrong diets, toxicity of drugs, or genetic predisposition. Fatty liver disease, as referred to herein, also includes the more severe forms thereof and, in particular, steatosis, non-alcoholic steatohepatitis (NASH) and non-alcoholic fatty liver disease (NAFLD).

In an embodiment, chronic liver disease is liver fibrosis or has liver fibrosis as a symptom. The term “liver fibrosis” is known in the art, as specified herein above. In an embodiment, liver fibrosis comprises excessive accumulation of extracellular matrix proteins, including collagen, in the liver. Said accumulation of extracellular matrix proteins can be verified e.g. by biopsy, e.g. as reviewed in Chowdhury and Mehta (2022), Clin Exp Med, doi.org/10.1007/s10238-022-00799-z. In an embodiment, liver fibrosis is accompanied by a reduced liver function. As referred to herein, liver fibrosis may be a symptom of liver cirrhosis, the latter term relating to a loss of liver function by excessive inflammation and/or scarring. Thus, chronic liver disease, in an embodiment, is advanced liver fibrosis, in an embodiment with a fibrosis level corresponding to METAVIR score stage F3 or F4, in an embodiment is sever/advanced fibrosis or cirrhosis. In an embodiment, liver fibrosis is a symptom of NASH and/or NAFLD. In an embodiment, liver fibrosis is a symptom of viral hepatitis, in particular HAV, HBV, HCV, HDV, and/or HEV hepatitis, all a specified herein above.

The term “subject”, as used herein, refers to a vertebrate animal, preferably a mammal and, more typically, to a human. In an embodiment, the subject is known or suspected to suffer from chronic liver disease, in an embodiment liver fibrosis. In an embodiment, the subject is suspected to suffer from and/or shows signs and symptoms of NASH and/or NAFLD. Thus, in an embodiment, the subject is suffering from non-viral chronic liver disease, in an embodiment from alcoholic steatohepatitis (ASH), non-alcoholic steatohepatitis (NASH), or non-alcoholic fatty liver disease (NAFLD). Such suspicion to suffer from chronic liver disease may in particular stem from preceding diagnostic measures, such as anamnesis, physical examination, ultrasound, radiography, MRT diagnostics, clinical chemistry diagnostics, and the like. The subject, in particular a subject known or suspected to suffer from chronic liver disease, may, however, also be a subject infected with a virus as specified herein above, in particular a hepatitis virus, in an embodiment HBV and/or HCV. In accordance, in an embodiment the subject is suffering from viral chronic liver disease, in an embodiment hepatitis C virus hepatitis and/or hepatitis B virus hepatitis. In a further embodiment, however, the subject does not suffer or is not known to suffer from a liver fibrosis, in an embodiment chronic liver disease, which is caused by or associated with alcohol abuse, viral infection as specified herein above, and/or autoimmune hepatitis as specified herein above; in a further embodiment, the patient is not suffering from hepatic viral etiologies, alcoholic steatohepatitis and/or hepatic autoimmune diseases.

The term “biomarker”, as used herein, refers to a molecular species which serves as an indicator for a disease or physiological state as referred to herein. Said molecular species can be a chemical compound which is detectable in a sample of a subject, in particular a metabolite of the subject's metabolism. Moreover, the biomarker may also be a molecular species which is derived from said metabolite. In such a case, the actual metabolite will be chemically modified in the sample or during the determination process and, as a result of said modification, a chemically different molecular species, i.e. the analyte, will be the determined molecular species. E.g., in case the biomarker is a polypeptide or protein, the analyte may be a derivative of the polypeptide or protein, may be a fragment of the polypeptide or protein, or may be a complex of the polypeptide, e.g. an immunocomplex of the polypeptide or a protein comprising more than one polypeptide. Also, in case the biomarker has an activity, e.g. a catalytic activity and/or an activating activity, e.g. on target cells, the biomarker may also be determined via said activity, e.g. in an enzymatic assay. It is to be understood that in the aforesaid cases, the analyte may represent the actual biomarker and has the same potential as an indicator for the respective medical condition as the biomarker would have. Preferred modes of determination and analytes for the biomarkers of the present description are described in the context of the respective biomarkers herein below. Moreover, as is understood by the skilled person, a biomarker according to the present invention need not necessarily correspond to one molecular species. Rather, the biomarker may comprise stereoisomers or enantiomers of a compound and/or, e.g. in case the biomarker is a polypeptide, may comprise variant molecular species, e.g. translated from splice variants, glycosylation variants, peptidase processing variants, and the like. In an embodiment, the variants share at least one determinable feature, e.g. an epitope or an activity.

The term “amount”, as used herein, includes any and all measure of quantity deemed suitable by the skilled person, and in particular includes an absolute amount of a compound referred to herein, a relative amount, or a concentration of the compound, as well as any value or parameter which correlates thereto or can be derived therefrom, in an embodiment by standard mathematical operations. Such values or parameters comprise intensity signal values from all specific physical or chemical properties obtained from the said compounds by direct measurements, e.g., intensity values in mass spectra or NMR spectra. Moreover, encompassed are all values or parameters which are obtained by indirect measurements specified elsewhere in this description, e.g., response levels determined from biological read out systems in response to the compounds or intensity signals obtained from specifically bound ligands, such as detection compounds. It is to be understood that values correlating to the aforementioned amounts or parameters can also be obtained by all standard mathematical operations. In the biomarker is an enzyme, such as glutamyltransferase (GGT), the term “amount” may also encompass or be the activity of the enzyme. Thus in a particular embodiment, the amount of glutamyltransferase (GGT) relates to the GGT activity.

The term “determining” as used herein refers to semiquantitative or quantitative determination of a biomarker referred to herein. Determining the amount of a biomarker may be carried out by any technique which allows for establishing a measure of quantity of a biomarker in a semiquantitative or quantitative manner. Suitable techniques depend on the molecular nature and the properties of the biomarkers and are discussed elsewhere herein in more detail.

Typically, the amount of a biomarker can be determined by determining a complex of the analyte with a detection compound, in particular an antibody or fragment thereof, i.e. in an immunoassay. Said determining of a complex of the analyte may be performed in any format deemed appropriate by the skilled person, in particular a sandwich, competition, or other assay format. Said assays will develop a signal which is indicative for the amount of a biomarker. Thus, determining may include micro-plate ELISA-based methods, fully-automated or robotic immunoassays (available, e.g., from Roche). Suitable measurement methods may also include precipitation (particularly immunoprecipitation), electrochemiluminescence (electro-generated chemiluminescence), RIA (radioimmunoassay), ELISA (enzyme-linked immunosorbent assay), fluorescent immunoassay (FIA), electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, or solid phase immune tests. Further methods known in the art such as gel electrophoresis, 2D gel electrophoresis, SDS polyacrylamid gel electrophoresis (SDS-PAGE) or Western Blotting. More typically, techniques particularly envisaged for determining the biomarkers referred to herein are described herein below.

The amount of a biomarker may in an embodiment be determined in an activity assay, in particular in case the biomarker has catalytic, e.g. enzymatic, or signaling activity. Enzyme assays for determining activity of clinically relevant enzymes are known in the art. Assays for determining signaling are also known in the art, e.g. IL-8 reporter gene assays.

In a further embodiment, the amount of a biomarker may be determined by detecting the amount of molecular species of the biomarker, or of fragments thereof. E.g., small molecule biomarkers may be detected as such or as their ions in mass spectrometry (MS). For polypeptide biomarkers detection of fragments thereof may be technically easier to put into practice. However, also other methods for detecting the amount of molecular species of the analyte are available, including chromatographic separation techniques such as liquid chromatography (LC), high performance liquid chromatography (HPLC), gas chromatography (GC), thin layer chromatography, and/or size exclusion or affinity chromatography, coupled to appropriate detection devices. Such a detection device may e.g. be a photometer, e.g. an UV/VIS-photometer or an MS device. Appropriate devices and methods are known in the art. Further suitable methods comprise measuring a physical or chemical property specific for the biomarker such as its precise molecular mass or NMR spectrum. Said methods comprise, preferably, biosensors, optical devices coupled to immunoassays, biochips, analytical devices such as mass-spectrometers, NMR-analyzers, surface plasmon resonance measurement equipment or chromatography devices.

The biomarkers to be determined in accordance with the present invention are as such known in the art. Moreover, methods for the determination of the amount of the biomarkers are known to the skilled person as well. For example, the biomarkers can be measured as described in the Examples section.

A biomarker of the present invention is Insulin-like growth factor-binding protein 3 (IGFBP3). IGFBP3 is a member of the family of Insulin-like growth factor-binding proteins known to the skilled person; the amino acid sequence of human IGFBP3 is e.g. shown in Genbank Acc No: EAL23801.1. The unglycosylated form of IGFBP3 has a molecular mass of 29 kDa; in biological samples, IGFBP3 may be complexed with Insulin-like growth factor I (IGF-1) and/or acid-labile subunit (ALS). In an embodiment, IGFBP3 is determined in an immunoassay, in a further embodiment a sandwich immunoassay. In an embodiment, IGFBP3 is determined in an electrochemiluminescence Immunoassay (ECLIA) using a capture anti-IGFBP3 antibody, which may e.g. biotinylated to mediate binding to a streptavidin-coated solid surface, and a detection anti-IGFBP3 antibody, which may be ruthenylated. The capture anti-IGFBP3 antibody and the detection anti-IGFBP3 antibody may comprise the same antibody or fragment thereof, e.g. a monoclonal antibody, an Fab fragment, or the like. In an embodiment, IGFBP3 is determined by the Elecsys IGFBP-3 Cobas® immunoassay manufactured by Roche Diagnostics GmbH, Mannheim. IGFBP3 may, however, also be determined by any other method deemed appropriate by the skilled person, in particular those described herein above.

A further biomarker of the present invention is gamma-glutamyltransferase (GGT), which is a known liver enzyme marker. In standard clinical chemistry tests the enzymatic activity of GGT is determined. The substrate used in enzymatic determination of the amount of GGT in an embodiment is a nitroanilide-conjugate of a gamma-glutamyl-peptide, in particular gamma-glutamyl-p-nitroanilide or L-gamma-glutamyl-3-carboxy-4-nitroanilide; the co-substrate (gamma-glutamyl acceptor) may in particular be glycylglycin. In an embodiment, GGT is determined by a method recommended by the International Federation of Clinical Chemistry (IFCC). In a further embodiment, GGT is determined by the GGT-2 Cobas® enzyme assay manufactured by Roche Diagnostics GmbH, Mannheim. GGT may, however, also be determined by any other method deemed appropriate by the skilled person, in particular those described herein above; the amino acid sequence of human GGT is e.g. shown in Genbank Acc. No: NP_001275762.1.

An optional further biomarker of the present invention is Interleukin-8 (IL-8). IL-8 is a member of the interleukin family of proteins known to the skilled person; the amino acid sequence of human IL-8 is e.g. shown in Genbank Acc No: AAH13615.1. In an embodiment, IL-8 is determined in an immunoassay, in a further embodiment a sandwich immunoassay. In an embodiment, IL-8 is determined in a florescence immunoassay (FIA) using a capture anti-IL-8 antibody, which may e.g. be bound to a solid surface, and a detection anti-IL-8 antibody, which may be conjugated to digoxygenin; in such case detection may be based on fluorescent-dye coupled latex beads further conjugated to anti-digoxygenin antibodies. In a further embodiment, IL-8 is determined by the IMPACT IL-8 immunoassay manufactured by Roche Diagnostics GmbH, Mannheim (Claudon et al. (2008), Clinical Chemistry 54(9):1463).

Further optional biomarkers of the invention, of which one or more may be determined in addition to IGFBP3, GGT, and, optionally, IL-8, are aspartate aminotransferase, alanine aminotransferase, platelet count, haptoglobin, alpha2-macroglobulin, apolipoprotein Al, bilirubin, cholesterol, hyaluronan, prothrombin index, hepatocyte growth factor (HGF), Tissue inhibitors of metalloproteinases (TIMPs), and/or urea. All these markers are as such known in the art and the skilled person knows how to select an appropriate determining method, in particular from those described herein above and or from standard methods. As will be understood from the description herein, the assessment may comprise further determining steps and/or other diagnostic measures, such as in particular determination of one or more further biomarker(s) not expressly referred to herein.

Aspartate aminotransferase (AST or ASAT) catalyzes the transamination from L-aspartate to a-ketoglutarate, forming L-glutamate and oxalacetate. The oxalacetate formed is reduced to malate by malate dehydrogenase (MDH) with simultaneous oxidation of reduced nicotinamide adenine dinucleotide (NADH). The change in absorbance with time due to the conversion of NADH to NAD is directly proportional to the AST activity and can be e.g. measured using a bichromatic (340, 700 nm) rate technique. Alanine aminotransferase (ALAT) catalyzes the transamination of L-alanine to a-ketoglutarate (α-KG), forming L-glutamate and pyruvate. The pyruvate formed is reduced to lactate by lactate dehydrogenase (LDH) with simultaneous oxidation of reduced nicotinamide-adenine dinucleotide (NADH). The change in absorbance is directly proportional to the alanine aminotransferase activity and can be, e.g., measured using a bichromatic (340, 700 nm) rate technique. Platelet count is the number of platelets per volume in a sample, typically a blood or plasma sample. Haptoglobin is a soluble plasma protein and may be determined e.g. in an immunoassay; the amino acid sequence of the human haptoglobin is e.g. provided in Genbank Acc. No: NP_001119574.1. Alpha2-macroglobulin is a soluble plasma protein and may be determined e.g. in an immunoassay; an amino acid sequence of the human alpha2-macroglobulin is e.g. provided in Genbank Acc. No: NP_000005.3. Apolipoprotein A1 is a plasma protein and may be determined e.g. in an immunoassay; an amino acid sequence of the human Apolipoprotein A1 is e.g. provided in Genbank Acc. No: NP_000030.1. HGF is a secreted paracrine cellular growth, motility and morphogenic factor, which may be determined e.g. in blood-derived samples, in particular. in an immunoassay; an amino acid sequence of the human HGF is e.g. provided in Genbank Acc. No: NP_000592.3. TIMPs are a family of inhibitors of metalloprotease activity involved in regulation of extracellular matrix (ECM) deposition and degradation and may be determined e.g. in an immunoassay; an exemplary amino acid sequence of a human TIMP is e.g. provided in Genbank Acc. No: CAA00898.1. Bilirubin, cholesterol, hyaluronan, prothrombin index, and urea are classical clinical chemistry markers and methods for their determination are known to the skilled person.

The term “sample”, as used herein, refers to a biological sample from a body fluid, in an embodiment, blood, plasma, serum, saliva or urine, or a sample derived from cells, tissues or organs, in particular from the liver, e.g., by biopsy. In a further embodiment, the sample is a blood, plasma or serum sample, in a further embodiment a serum or plasma sample. Biological samples can be derived from a subject by techniques known in the art. For example, blood samples may be obtained by blood taking, while tissue or organ samples are to be obtained, e.g., by biopsy. In an embodiment, the sample is known or suspected to comprise biomarkers referred to herein. The aforementioned samples may be pre-treated before they are used for according to the present invention. Said pre-treatment may include treatments required to release or separate the biomarker(s) and/or the analyte(s) or to remove excessive material or waste. Suitable techniques comprise centrifugation, extraction, fractioning, ultrafiltration, protein precipitation followed by filtration and purification and/or enrichment of compounds.

Moreover, other pre-treatments may be carried out in order to provide the biomarker and/or analyte in a form or concentration suitable for the intended determination. Suitable and necessary pre-treatments depend on the means used for carrying out the method of the invention and are well known to the person skilled in the art. Pre-treated samples as described before are also comprised by the term “sample” as used in accordance with the present invention.

The term “reference”, as used herein, relates to a value, e.g. an amount or any value derived therefrom, e.g. a score, which can be correlated to a medical condition and, in an embodiment, which allows for the assessment of the invention to be made, in a further embodiment enables allocation of a subject into either a group of subjects suffering from a disease or condition or being at risk for developing it, or a group of subjects which do not suffer from said disease or condition or which are not at risk for developing it. Such a reference can be a threshold value, e.g. a threshold amount, which separates these groups from each other. Accordingly, the reference may be a value which allows for allocation of a subject into a group of subjects suffering from a disease or condition or being at risk for developing it, or not. For example, the reference may be a value which allows for allocation of a subject into a group of subjects suffering from chronic liver disease, or not being at risk of developing chronic liver disease. The reference may, however, also be a reference range, e.g., in an embodiment, a range of values for which chronic liver disease can be excluded. Furthermore, the reference may be a value calculated from the aforesaid values, e.g. from the amounts of two or more biomarkers, in an embodiment to provide a score. A suitable reference separating the two groups can be provided without further ado e.g. by the statistical tests referred to herein elsewhere based on values of biomarkers from suitable reference groups as specified herein below. As the skilled person understands, it may not always be possible, although particularly envisaged, to provide a reference unambiguously allocating each and every possible value of a biomarker to one of the aforesaid groups; thus, there may be a range of values for which a clear assessment cannot be provided. In an embodiment, however, as indicated above, a reference enables the assessment to be made for each and every value of a biomarker or set of biomarkers which may be measured. As the skilled person understands, the specific value of a reference may depend on the assessment intended and on parameters thereof; thus, the reference value for assessing chronic liver disease may typically different from the reference value for assessing e.g. severe liver fibrosis. Relevant parameters having an influence on the reference may in particular be sensitivity and specificity of assessment, as illustrated e.g. in the Examples.

As indicated herein above, a reference may in particular be derived from at least one reference group, the term “reference group” relating to a group of subjects with known status with regard to the assessment. Thus the reference group may e.g. be a group of subjects for which it is known whether they suffer from chronic liver disease. The population of subjects in a reference group in an embodiment comprises a plurality of subjects, e.g. at least 5, 10, 50, 100, 1,000, or 10,000 subjects. Typically, the subject to be diagnosed and the subjects of the said reference group are of the same species. The reference applicable for an individual subject may vary depending on various physiological parameters such as age, gender, or subpopulation. As is understood by the skilled person, prevalence of chronic liver disease in the population is low, in an embodiment less than 2%; thus, a reference may be derived also from the average population. Assuming that contribution of actually afflicted subjects is low, such an average population reference group may be treated as a reference group known not to suffer from chronic liver disease; in an embodiment, in such case, the size of the reference group is sufficiently high, e.g. at least 100, in a further embodiment at least 1000, in a further embodiment at least 10000 subjects. In view of the description herein, the skilled person understands that a reference group may, in principle, also be a mixed population of subjects with regard to chronic liver disease, provided that the status of each member of said mixed population with regards to chronic liver disease is or becomes known before deriving a reference from such group.

Reference amounts can, in principle, be calculated for a cohort of subjects based on the average or mean values for a given parameter such as biomarker amount by applying standard statistically methods. In particular, accuracy of a test such as a method aiming to diagnose an event, or not, is best described by its receiver-operating characteristics (ROC) (see especially Zweig 1993, Clin. Chem. 39:561-577). The ROC graph is a plot of all of the sensitivity/specificity pairs resulting from continuously varying the decision threshold over the entire range of data observed. The clinical performance of a diagnostic method depends on its accuracy, i.e. its ability to correctly allocate subjects to a certain prognosis or diagnosis. The ROC plot indicates the overlap between the two distributions by plotting the sensitivity versus 1-specificity for the complete range of thresholds suitable for making a distinction. On the y-axis is sensitivity, or the true-positive fraction, which is defined as the ratio of number of true-positive test results to the product of number of true-positive and number of false-negative test results. This has also been referred to as positivity in the presence of a disease or condition. It is calculated solely from the affected subgroup. On the x-axis is the false-positive fraction, or 1-specificity, which is defined as the ratio of number of false-positive results to the product of number of true-negative and number of false-positive results. It is an index of specificity and is calculated entirely from the unaffected subgroup. Because the true-and false-positive fractions are calculated entirely separately, by using the test results from two different subgroups, the ROC plot is independent of the prevalence of the event in the cohort. Each point on the ROC plot represents a sensitivity/-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. If the ROC plot falls completely below the 45° diagonal, this is easily remedied by reversing the criterion for “positivity” from “greater than” to “less than” or vice versa. Qualitatively, the closer the plot is to the upper left corner, the higher the overall accuracy of the test. Dependent on a desired confidence interval, a threshold can be derived from the ROC curve allowing for the diagnosis or prediction for a given event with a proper balance of sensitivity and specificity, respectively. Accordingly, the reference to be used for the aforementioned method of the present invention, i.e. a threshold which allows to discriminate between subjects being at risk and not being at risk can be generated, usually, by establishing a ROC for said cohort as described above and deriving a threshold amount therefrom. Dependent on a desired sensitivity and specificity for a diagnostic method, the ROC plot allows deriving suitable thresholds. It will be understood that an optimal sensitivity may be desired for excluding a subject for being at increased risk (i.e. a rule-out), whereas an optimal specificity may be envisaged for a subject to be assessed as being at an increased risk (i.e. a rule-in).

The term “comparing” as used herein encompasses comparing the determined amount for a biomarker as referred to herein to a reference. It is to be understood that comparing as used herein refers to any kind of comparison made between the value for the amount with the reference. However, it is to be understood that, in an embodiment, identical types of values are compared with each other, e.g., if an absolute amount is determined, the reference shall also be an absolute amount, if a relative amount is determined, the reference shall also be a relative amount, etc. The term comparing also encompasses comparing a calculated score with a suitable reference core. The comparison may be carried out manually or computer assisted. The value of the amount and the reference can be, e.g., compared to each other and the said comparison can be automatically carried out by a computer program executing an algorithm for the comparison. The computer program carrying out the said evaluation will provide the desired assessment in a suitable output format. As set forth above, it is also envisaged to calculate a score based on the amounts of the biomarkers, in particular a single score, and to compare this score to a reference score. The calculated score in an embodiment combines information on the amounts of the biomarkers. Moreover, in the score, the biomarkers may be weighted in accordance with their contribution to the establishment of the differentiation, wherein the weighting factor of the individual biomarkers may be different. The score can be regarded as a classifier parameter for the assessing as set forth herein. In particular, it enables providing the assessment based on a single score. Thus, the skilled person does not have to interpret the entire information on the amounts of the individual biomarkers. Using a scoring system as described herein, values of different dimensions or units for the biomarkers may be used since the values will be mathematically transformed into the score. Accordingly, e.g. values for absolute concentrations may be combined in a score with peak area ratios and/or enzymatic activity values. The reference score to be applied may be elected based on the desired sensitivity and/or the desired specificity. How to elect a suitable reference score is well known in the art.

The method for assessing chronic liver disease comprises step (a) determining an amount of the biomarker Insulin-like growth factor-binding protein 3 (IGFBP3) in a sample from said subject. The biomarker IGFBP3 and methods for determining an amount thereof have been described herein above. In an embodiment, the amount of IGFBP3 is determined in a blood derived sample, in a further embodiment a serum or plasma sample. In an embodiment, the amount of IGFBP3 is determined as a concentration; to the value obtained, standard mathematical and statistical operations may be applied, such as standardization, normalization, log-transformation, e.g. log10 transformation, and the like. The IGFBP3 concentration typically decreases in subjects suffering from chronic liver disease, in particular METAVIS stage F3 and F4 fibrosis and cirrhosis. In an embodiment, IGFBP3 may be used independently of disease etiology.

The method for assessing chronic liver disease comprises step (b) determining an amount of the biomarker gamma-glutamyltransferase (GGT) in the sample. The biomarker GGT and methods for determining an amount thereof have been described herein above. In an embodiment, the amount of GGT is determined in a blood derived sample, in a further embodiment a serum or plasma sample. In an embodiment, the amount of GGT is determined as an enzymatic activity, as specified herein above; to the value obtained, standard mathematical and statistical operations may be applied, such as standardization, normalization, log-transformation, e.g. log10 transformation, and the like. The GGT activity typically increases in subjects suffering from chronic liver disease, in particular METAVIS stage F3 and F4 fibrosis and cirrhosis.

The method for assessing chronic liver disease comprises step (c) comparing the amounts of the biomarkers determined in steps (a) and (b) to references for said biomarkers and/or calculating a score for assessing chronic liver disease. References have been described herein above. As the skilled person understands, in case one or more further biomarkers are determined, these are in an embodiment compared to references and/or are included in calculating the score as well. As is also understood by the skilled person, parameters determined for amounts of biomarkers are typically compared to corresponding references; thus, in case a concentration is determined e.g. in step (a), said concentration is compared to a reference concentration; and/or in case an activity is determined e.g. in step (b), said activity is compared to a reference activity. In a further embodiment, in case a score is calculated from the amounts determined in steps (a) and (b), said score is compared to a reference score calculated from reference mounts of the biomarkers IGFBP3 and GGT by the same mathematical operations. In an embodiment, to calculate a score, the amounts of the biomarkers determined may be linearly combined (e.g. Score=c₀+c₁*[Biomarker₁]+c₂*[Biomarker₂]+. . . ), preferably with corresponding coefficients (c₁, c₂, . . . ) and intercept (c₀). In preferred embodiments, the amounts of the biomarkers ([Biomarker₁], [Biomarker₂], . . . ) are log transformed (e.g. log 2 or log 10, preferably log 10) for the linear combination to a score. The output of such a linear combination may be directly used as score. Alternatively or additionally, the output of this linear combination may be used as input to a mathematical transformation (e.g. a sigmoid transformation such as (f(x)=1/(1+exp(−x)))) providing a risk score, which maps the output range of the score to 0-1. The score may be proportional to the risk for chronic liver disease (e.g., the score may increase with increased liver fibrosis stage).

The specific comparison made in an embodiment depends on the specific reference(s) used. In case the reference is a threshold, the comparison may comprises determining whether the value of the sample in question is beyond said threshold; in case the reference is a reference range, the comparison may comprise establishing whether there value of the sample in question is within the reference range, or not.

The method for assessing chronic liver disease comprises step (d) assessing chronic liver disease in said subject based on the comparison and/or the calculation made in step (c). The term “assessing” has been specified herein above. Typically, the assessment in step (d) may in particular be based on the status of the reference group(s) and the reference derived therefrom. As the skilled person understands, a biomarker may be decreased or increased in an afflicted subject compared to a healthy reference. Thus, in case the reference is derived from a group of subjects known to be afflicted with the disease or condition, a biomarker value essentially identical to the reference in an embodiment leads to an assessment of the subject under investigation being afflicted with the disease or condition, and a value different, in an embodiment significantly different, from said reference in a further embodiment leads to an assessment of the subject under investigation not being afflicted with the disease or condition. Conversely, in case the reference is derived from a group of subjects known not to be afflicted with the disease or condition, a biomarker value essentially identical to the reference in an embodiment leads to an assessment of the subject under investigation not being afflicted with the disease or condition, and a value different, in an embodiment significantly different, from said reference in a further embodiment leads to an assessment of the subject under investigation being afflicted with the disease or condition. Further, as indicated above, the reference may be a threshold value; such a threshold value may be derived from a reference group known to suffer from the disease of condition, from a reference group known not to suffer from the disease of condition, or from a reference group known to suffer from the disease of condition and a reference group known not to suffer from the disease of condition. Typically, in case a biomarker value of a subject under investigation exceeds the aforesaid threshold reference toward the values of the reference group known to suffer from the disease or condition, it will be assumed that the subject suffers from the disease of condition; and in case a biomarker value of a subject under investigation exceeds the aforesaid threshold toward the values of the reference group known to not suffer from the disease or condition, it will be assumed that the subject does not suffer from the disease or condition. Thus, depending on the specific biomarker and its correlation with a disease or condition, values found in a sample which are higher than or equal to a threshold may be indicative for the presence of a medical condition while those being lower may be indicative for the absence of the medical condition; or, it may also be that values found in a sample to be investigated which are lower or identical than the threshold are indicative for the presence of a medical condition while those being higher are indicative for the absence of the medical condition. As the skilled person understands in view of the description herein, the above applies mutatis mutandis to a score, which may incorporate amounts of more than one biomarker, in an embodiment all biomarkers. As specified herein above, a reference score may in particular be calculated by the same mathematical operations applied to calculate a score, but using values from one or more reference group(s). The reference score also may be e.g. a threshold score or a reference score range. As the skilled person will understand as well, the assessment following from the comparison of a score to a reference score will depend on the specificities of score calculation; thus, whether a score above or below a threshold score is indicative of chronic liver disease will depend on the specific way of calculating the score. E.g. in the exemplary score calculated according to the Examples, a score higher than the cutoff score is indicative of chronic liver disease. In an embodiment, assessing is differentiating NASH fibrosis from NAFLD and/or absence of chronic liver disease.

The result of the assessment in step (d), in an embodiment, is a statement concerning the status of the subject with regards to chronic liver disease. Said result may be implicit, e.g. by juxtaposing the value determined in the sample to one or more reference(s), which may be further evaluated, e.g. by a medical practitioner. The result may, however, also be explicit, e.g. by annunciating that the comparison suggests a specific status with regards to chronic liver disease. Thus, the method may further comprise a step of annunciating the result of the assessment of step (d). Alternatively or in addition, the result of the assessment in step (d) may also be used in further assessments, e.g. by inclusion into or combination with results of further assessments, e.g. further diagnostic measures, such as sonography, magnetic resonance imaging, radiography, transient elastography, and/or determining subject age and/or gender.

The result of the assessment is step (d) may form the basis for a treatment of the subject or a decision thereon. Thus, in case it is in an embodiment assessed that the subject suffers from chronic liver disease, said subject may be treated for chronic liver disease. Appropriate treatments are known in the art and include in particular therapy of underlying disease, such as viral infection, metabolic disease and/or diabetes, adipositas, hemochromatosis and/or Wilson disease. Thus, treatment may in particular comprise recommending lifestyle change, in an embodiment may comprise treatment to reduce body weight, to improve nutrition, and/or to avoid or reduce intoxicants; treatment may also comprise antiviral treatment, such as administration of at least one nucleotide analog, interferon, and/or Entecavir, in particular in case of HBV infection; and/or administration of direct-acting antiviral agents (DAAs), in particular in case of HCV infection. In an embodiment, said treatment comprises administration of at least one compound independently selected from the group consisting of an FXR agonist, a PPAR agonist, a dual CCR2 and CCR5 antagonist, an FGF19 analog, an FGF21 analog, an apoptosis signal-regulating kinase 1 inhibitor, a pan-caspase inhibitor, a TGF-beta inhibitor, an anti-LOXL2 antibody, an angiotensin receptor blocker, and a combined angiotensin receptor and ACE enzyme inhibitor. Corresponding compounds are known in the art and are reviewed e.g. in Guo and Lu (2020), J Cin Transl Hepatol 8:304.

Advantageously, it was found in the work underlying the present invention that the easily accessible biomarkers described herein allow diagnosis of chronic liver disease and various physiological states related thereto with improved reliability, allowing improved monitoring of disease and assignment of patients to treatment options.

The definitions made above apply mutatis mutandis to the following. Additional definitions and explanations made further below also apply for all embodiments described in this specification mutatis mutandis.

The present invention further relates to an, in an embodiment computer-implemented, method for assessing chronic liver disease in a subject, said method comprising
(A) obtaining an amount of the biomarker Insulin-like growth factor-binding protein 3 (IGFBP3) in a sample from said subject;
(B) obtaining an amount of the biomarker gamma-glutamyltransferase (GGT) in said sample;
(C) comparing the amounts of the biomarkers determined in steps (a) and (b) to references for said biomarkers and/or calculating a score for assessing chronic liver disease; and
(D) assessing chronic liver disease in said subject based on the comparison and/or the calculation made in step (c).

The aforesaid method for assessing chronic liver disease, in an embodiment, is an in vitro method, in a further embodiment is an in silico method. Thus, the aforesaid method may in particular be a method carried out by a computer comprising the steps described herein above. Moreover, the method may comprise steps in addition to those explicitly mentioned above. For example, further steps may relate, e.g., to determining an amount of a biomarker for step (A) and/or (B), in an embodiment as specified herein above; and/or annunciating and/or otherwise further using the result of the assessment after step (D). Moreover, one or more of said steps may be assisted or performed by automated equipment.

The term “obtaining”, as used herein, relates to acquiring the indicated information, in particular a value of a parameter such as an amount of a biomarker, in a manner enabling basing the assessment on said information. Thus, in an embodiment, obtaining is reading the information from a data carrier, e.g. in the form of a data sheet, analysis device output, e.g. a result of an immunoassay, a mass spectrum, or the like; or from a database comprising at least the relevant information. In an embodiment, the information obtained comprises amounts of biomarkers determined as specified herein above. The data carrier and/or the database may be local, i.e. physically connected to a device used to perform steps of the method; they may, however, also be remote, accessible via a network connection or via the internet; thus, the data carrier and/or the data carrier may e.g. be cloud storage. Also, the method may be implemented as a service provided by means of a network connection, e.g. via internet, in which values of the biomarkers, or one or more score(s) derived therefrom, are obtained from e.g. a device performing the determination or medical practitioner, and the result of the comparison it output.

The term “computer-implemented”, as used herein, means that the method is carried out in an automated fashion on a data processing unit which is, typically, comprised in a computer or similar data processing device. The data processing device shall receive values for the amount of the biomarkers. Such values can be the amounts, relative amounts or any other calculated value reflecting the amount as described elsewhere herein in detail. Accordingly, it is to be understood that the aforementioned method does not necessarily require the determination of amounts for the biomarkers but rather may use values for already predetermined amounts.

The present invention also relates to a database comprising stored references for the biomarkers IGFBP3 and GGT for assessing chronic liver disease.

The term “database”, as used herein, refers to a collection of data which may be physically and/or logically grouped together. Accordingly, the database in an embodiment comprises an allocation of references to assessment results. As the skilled person understands from the description herein above, “stored references for the biomarkers IGFBP3 and GGT” may also be one or more scores derived from said references. The database, in an embodiment, comprises further data, such as upper and/or lower detection limits, references for further biomarkers, in particular those described herein above, data relevant for plausibility checks, and the like. In a further embodiment, the database comprises data on one or more assay methods to use, lot-specific data, e.g. for calibrator samples, and the like. In an embodiment, the database may be implemented in a single data storage medium or in physically separated data storage media being operatively linked to each other. In an embodiment, the database comprises a data collection on a suitable storage medium, in an embodiment tangible embedded thereon. Moreover, the database, in an embodiment, further comprises a database management system. The database management system is, in an embodiment, a network-based, hierarchical or object-oriented database management system. Furthermore, the database may be a federal or integrated database. In a further embodiment, the database will be implemented as a distributed (federal) system, e.g. as a Client-Server-System. In a further embodiment, the database is structured as to allow a search algorithm to compare a test data set with the data sets, in particular the references, comprised by the data collection. Specifically, by using such an algorithm, the database can be searched for similar or identical data sets being indicative for a medical condition or effect as set forth above (e.g. a query search). Thus, in an embodiment, if data set fulfilling the comparison criteria as detailed elsewhere herein can be identified in the database, the test data set will be associated with the said medical condition or effect. Consequently, the information obtained from the database can be used, e.g., as a reference for the methods described elsewhere herein.

The present invention further relates to a device comprising
(a) at least one measuring unit for determining an amount of a first biomarker being IGFBP3 and an amount of a second biomarker being GGT in a sample of the subject, said at least one measuring unit comprising at least one detection means for the first biomarker and the second biomarker; and
(b) an evaluation unit operably linked to the measuring unit, said evaluation unit comprising a data processor comprising instructions for carrying out a comparison of the amount of the first biomarker and the second biomarker to references and/or for carrying out a calculation of a score for assessing based on the amounts of the biomarkers.

The term “device” as used herein relates to a combination of means comprising the aforementioned units operatively linked to each other as to allow the determination of the amounts of biomarkers and evaluation thereof according to a method as specified herein such that an assessment can be provided. The device comprises at least one measuring unit and at least one evaluation unit.

The analyzing unit, typically, comprises at least one reaction zone having a first detection agent for the first biomarker and a second detection agent for the second biomarker. The device may comprise one or more further detection agent(s) for one or more further, optional biomarker(s). The detection agent may be comprised in immobilized form on a solid support or carrier which is to be contacted to the sample. The detection agent may, however, also be comprised e.g. in liquid form in the device, e.g. as a stock solution, in particular in case the biomarker is determined in an activity assay. Moreover, in the reaction zone, it is in an embodiment possible to apply conditions which allow for the specific binding of the detection agent(s) to the biomarkers comprised in the sample and/or for a catalytic reaction to occur. The reaction zone may either allow directly for sample application or it may be connected to a loading zone where the sample is applied. In the latter case, the sample can be actively or passively transported via a connection between the loading zone and the reaction zone to the reaction zone. Moreover, the reaction zone shall be also connected to a detector. The connection shall be such that the detector can detect the result of a detection reaction, e.g. the binding of the biomarkers to their detection agents or an enzymatic reaction. Suitable detectors depend on the techniques used for measuring the presence or amount of the biomarkers. For example, for optical detection, transmission of light may be required between the detector and the reaction zone while for electrochemical determination a fluidal connection may be required, e.g., between the reaction zone and an electrode. The detector shall be adapted to allow determination of the amount of the biomarker(s). The determined amount can be subsequently transmitted to the evaluation unit.

The evaluation unit comprises at least one data processor, which may also be referred to as a data processing unit, such as a computer, with an implemented algorithm for determining the amount present in the sample. Appropriate data processing units are known in the art and include in particular a Central Processing Unit (CPU), a Graphics Processing Units (GPU), an Application Specific Integrated Circuit (ASIC), a Tensor Processing Unit (TPU), a field-programmable gate array (FPGA), and other data processing units know in the art. A data processor may e.g. be a general purpose computer or a portable computing device. It should also be understood that multiple computing devices may be used together, such as over a network or other methods of transferring data, for performing one or more steps of the methods disclosed herein. Exemplary computing devices include desktop computers, laptop computers, personal data assistants (“PDA”), cellular devices, smart or mobile devices, tablet computers, servers, and the like. In general, a data processing unit comprises a processor capable of executing a plurality of instructions (such as a program of software).

The evaluation unit, typically comprises or has access to a memory. A memory is a computer readable information storage medium and may comprise a single storage device or multiple storage devices, located either locally with the computing device or accessible to the computing device across a network, for example. Computer-readable media may be any available media that can be accessed by the computing device and includes both volatile and non-volatile media. Further, computer readable-media may be one or both of removable and non-removable media. By way of example, and not limitation, computer-readable media may comprise computer storage media. Exemplary computer storage media includes, but is not limited to, RAM, ROM, EEPROM, flash memory or any other memory technology, CD-ROM, Digital Versatile Disk (DVD) or other optical disk storage, magnetic cassettes, magnetic tape, magnetic disk storage or other magnetic storage devices, or any other medium which can be used for storing a plurality of instructions capable of being accessed by the computing device and executed by the processor of the computing device. The evaluation unit, in an embodiment, further comprises a database as specified herein above.

According to embodiments of the instant disclosure, software may include instructions which, when executed by a processor of the computing device, may perform one or more steps of the methods disclosed herein. Some of the instructions may be adapted to produce signals that control operation of other units of the device, e.g. a measuring unit and/or other devices and thus may operate through those control signals to transform materials far removed from the device itself. These descriptions and representations are the means used by those skilled in the art of data processing, for example, to most effectively convey the substance of their work to others skilled in the art.

The plurality of instructions may also comprise an algorithm which is generally conceived to be a self-consistent sequence of steps leading to a desired result. These steps may include those requiring physical manipulations of physical quantities. Usually, though not necessarily, these quantities take the form of electrical or magnetic pulses or signals capable of being stored, transferred, transformed, combined, compared, and otherwise manipulated. It proves convenient at times, principally for reasons of common usage, to refer to these signals as values, characters, display data, numbers, or the like as a reference to the physical items or manifestations in which such signals are embodied or expressed. It should be borne in mind, however, that all of these and similar terms are to be associated with the appropriate physical quantities and are merely used here as convenient labels applied to these quantities.

The evaluation unit may also comprise or have access to an output device. Exemplary output devices include displays, printers, files, and telecommunication devices, such as fax devices, data servers, and the like. According to some embodiments, a computing device may perform one or more steps of a method disclosed herein, and thereafter provide an output, via an output device, relating to an assessment result, indication, ratio or other factor of the method.

The term “detection agent”, for which also “detection compound” may be used, as used herein, refers to any agent which allows determination, in an embodiment specific determination, of an amount of at least one biomarker. Thus, the detection agent may in particular be a reaction substrate, e.g. in case the biomarker has catalytic activity, e.g. is an enzyme; or the detection agent may be an agent binding, in an embodiment specifically, to a biomarker or analyte thereof.

The skilled person selects suitable detection substrates in dependence on the biomarker, i.e. catalytic activity, to be detected, based on information available in the art. For specific biomarkers, example substrates are provided herein above.

Also, binding agents for specific antigens, as well as methods for providing them, are known in the art. As indicated herein above, the detection agent being a binding agent in an embodiment specifically binds to a biomarker, i.e. does not cross-react with other components present in the sample. Typically, a detection agent specifically binding a biomarker as referred to herein may be an antibody, an antibody fragment or derivative, an aptamer, a ligand for the biomarker, a receptor for the biomarker, an enzyme known to bind and/or convert the biomarker, or a small molecule known to specifically bind to the biomarker. For example, antibodies as referred to herein as detection agents include both polyclonal and monoclonal antibodies, as well as fragments thereof, such as Fv, Fab and F(ab)2 fragments that are capable of binding antigen or hapten. Aptamer detection agents, e.g., may be nucleic acid or peptide aptamers. Methods to prepare such aptamers are well-known in the art. The detection agent may be fused or linked permanently or reversibly to a detectable label. Suitable labels are well known to the skilled artisan. Suitable detectable labels are any labels detectable by an appropriate detection method. Typical labels include gold particles, latex beads, acridan ester, luminol, ruthenium, enzymatically active labels, radioactive labels, magnetic labels (“e.g. magnetic beads”, including paramagnetic and superparamagnetic labels), and fluorescent labels.

“Specific binding” of a detection agent means that it should not bind substantially to, i.e. cross-react with, another peptide, polypeptide or substance present in the sample to be analyzed. Preferably, the specifically bound biomarker should be bound with at least 3 times higher, more preferably at least 10 times higher and even more preferably at least 50 times higher affinity than any other components of the sample. Non-specific binding may be tolerable, if it can still be distinguished and measured unequivocally, e.g. according to its size on a Western Blot, or by its relatively higher abundance in the sample.

The present invention also relates to a kit for assessing chronic liver disease in a subject comprising a first detection agent for determining an amount of IGFBP3 and second detection agent for determining an amount of GGT.

The term “kit” as used herein refers to a collection of the aforementioned components, typically, provided in separate compound(s) or as a mixture of compounds. The means are, in an embodiment, provided in a single container (i.e. a housing), in a further embodiment enabling common translocation, e.g. transport, of the components. The container also typically comprises instructions for carrying out the method of the present invention. These instructions may be in the form of a manual or may be provided by a computer program code which is capable of carrying out or supports the determination of the biomarkers referred to in the methods of the present invention when implemented on a computer or a data processing device. The computer program code may be provided on a data storage medium or device such as an optical storage medium (e.g., a Compact Disc) or directly on a computer or data processing device or may be provided in a download format such as a link to an accessible server or cloud. Moreover, the kit may, usually, comprise standards for reference amounts of biomarkers for calibration purposes. The kit may also comprise further components which are necessary for carrying one of the methods described herein, may assist in doing so, or may provide further functions; in an embodiment, the further component is a solvent, a buffer, a diluent, a washing solution and/or one or more reagent(s) required for detection of a biomarker. Further, the kit may comprise the device of the invention either in parts or in its entirety.

The present invention also relates to a method for assessing and treating chronic liver disease, said method comprising the steps of a method for assessing according to the present invention and the further step of treating said chronic liver disease in a subject identified to suffer therefrom.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, as specified herein above. Exemplary treatments envisaged have been described herein above.

As referred to herein, treating also includes preventing deterioration of disease, i.e. preventing aggravation. The term “preventing aggravation” refers to retaining a given health status with respect to the diseases or disorders referred to herein for a certain period of time in a subject. It will be understood that said period of time may be dependent on the type and quantity of treatment which has been administered and individual factors of the subject discussed elsewhere in this specification. It is to be understood that prevention of aggravation may not be effective in all subjects treated. However, the term requires that, in an embodiment, a statistically significant portion of subjects of a cohort or population are effectively prevented from aggravation of a disease or disorder referred to herein or its accompanying symptoms. In an embodiment, a cohort or population of subjects is envisaged in this context which normally, i.e. without preventive measures, would develop aggravation of a disease or disorder as referred to herein. Whether a portion is statistically significant can be determined without further ado by the person skilled in the art using various well known statistic evaluation tools discussed elsewhere in this specification.

The present invention further relates to a use of (i) a first biomarker being IGFBP3 and a second biomarker being GGT and/or (ii) a first detection agent for determining an amount of IGFBP3 and a second detection agent for determining an amount of GGT, for assessing chronic liver disease; and to a use of a first detection agent for determining an amount of IGFBP3 and a second detection agent for determining an amount of GGT for the manufacture of a diagnostic for assessing chronic liver disease.

The invention further discloses and proposes a computer program including computer-executable instructions for performing the method according to the present invention in one or more of the embodiments enclosed herein when the program is executed on a computer or computer network or a device as specified herein. Specifically, the computer program may be stored on a computer-readable data carrier. Thus, specifically, one, more than one or even all of method steps a) to d) as indicated above may be performed by using a computer or a computer network, in an embodiment by using a computer program. Thus, the present invention in particular proposes a computer program comprising instructions which, when the program is executed by a computer, cause the computer to carry out a method as specified herein above; and to a computer-readable medium comprising instructions which, when executed by a computer, cause the computer to carry out a method as specified herein above; to a computer-readable data carrier having stored thereon the computer program as specified herein above; and to a data carrier signal carrying the computer program as specified herein above. The present invention also relates to a data processing apparatus, device, or system comprising means for carrying out performing the method according to the present invention; to a data processing apparatus, device, or system comprising a processor configured to perform the method according to the present invention.

The invention further discloses and proposes a computer program product having program code means, in order to perform the method according to the present invention in one or more of the embodiments enclosed herein when the program is executed on a computer or computer network. Specifically, the program code means may be stored on a computer-readable data carrier.

Further, the invention discloses and proposes a data carrier having a data structure stored thereon, which, after loading into a computer or computer network, such as into a working memory or main memory of the computer or computer network, may execute the method according to one or more of the embodiments disclosed herein.

The invention further proposes and discloses a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier. Specifically, the computer program product may be distributed over a data network.

Finally, the invention proposes and discloses a modulated data signal which contains instructions readable by a computer system or computer network, for performing the method according to one or more of the embodiments disclosed herein.

In an embodiment, referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, the present invention further discloses:
A computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

Summarizing the findings of the present invention, the following embodiments are particularly envisaged:

Embodiment 1: A method for assessing chronic liver disease in a subject, said method comprising
(a) determining an amount of the biomarker Insulin-like growth factor-binding protein 3 (IGFBP3) in a sample from said subject;
(b) determining an amount of the biomarker gamma-glutamyltransferase (GGT) in said sample;
(c) comparing the amounts of the biomarkers determined in steps (a) and (b) to references for said biomarkers and/or calculating a score for assessing chronic liver disease; and (
d) assessing chronic liver disease in said subject based on the comparison and/or the calculation made in step (c).

Embodiment 2: The method of embodiment 1, wherein said chronic liver disease is liver fibrosis.

Embodiment 3: The method of embodiment 1 or 2, wherein said chronic liver disease is advanced liver fibrosis, in an embodiment corresponding to METAVIR score stage F3 or F4, or cirrhosis.

Embodiment 4: The method of any one of embodiments 1 to 3, wherein said assessing comprises diagnosing liver fibrosis, in an embodiment comprises diagnosing advanced liver fibrosis.

Embodiment 5: The method of any one of embodiments 1 to 4, wherein said assessing comprises staging chronic liver disease.

Embodiment 6: The method of any one of embodiments 2 to 5, wherein said assessing comprises staging liver fibrosis.

Embodiment 7: The method of any one of embodiments 1 to 6, wherein said assessing comprises differentiating between non-advanced liver fibrosis and advanced liver fibrosis. Embodiment 8: The method of any one of embodiments 1 to 7, wherein said assessing comprises excluding advanced liver fibrosis, in an embodiment comprises excluding liver fibrosis.

Embodiment 9: The method of any one of embodiments 1 to 8, wherein said method is comprised in a method of monitoring chronic liver disease.

Embodiment 10: The method of any one of embodiments 1 to 9, wherein said method further comprises the steps of determining an amount of the biomarker Interleukin-8 (IL-8), and wherein step (c) comprises comparing the amounts of the three biomarkers determined to references for said biomarkers and/or calculating a score for assessing chronic liver disease.

Embodiment 11: The method of any one of embodiments 1 to 10, wherein the amount of the biomarker IGFBP3 and optionally the amount of the biomarker IL-8 is/are determined by an immunoassay.

Embodiment 12: The method of any one of embodiments 1 to 11, wherein the amount of the biomarker GGT is determined by an activity assay.

Embodiment 13: The method of any one of embodiments 1 to 12, wherein said subject is known or suspected to suffer from liver disease, in an embodiment from chronic liver disease.

Embodiment 14: The method of any one of embodiments 1 to 13, wherein said subject is suffering from viral chronic liver disease, in an embodiment hepatitis C virus hepatitis and/or hepatitis B virus hepatitis.

Embodiment 15: The method of any one of embodiments 1 to 14, wherein said subject is suffering from non-viral chronic liver disease, in an embodiment from alcoholic steatohepatitis

(ASH), non-alcoholic steatohepatitis (NASH), or non-alcoholic fatty liver disease (NAFLD). Embodiment 16: The method of any one of embodiments 1 to 15, wherein said subject is a human.

Embodiment 17: The method of any one of embodiments 1 to 16, wherein said sample is a bodily fluid sample, in an embodiment a blood sample or a blood-derived sample, in a further embodiment a blood, plasma, or serum sample, in a further embodiment a plasma or serum sample.

Embodiment 18: The method of any one of embodiments 1 to 17, wherein said references are references for each biomarker derived from at least one subject known to suffer from chronic liver disease, in an embodiment from liver fibrosis, in a further embodiment from advanced liver fibrosis.

Embodiment 19: The method of embodiment 18, wherein amounts for each of the biomarkers being essentially identical or similar to the corresponding references are indicative for a subject suffering from chronic liver disease, in an embodiment from liver fibrosis, in a further embodiment from advanced liver fibrosis; and/or amounts for each of the biomarkers being different from the corresponding references are indicative for a subject not suffering from chronic liver disease, in an embodiment from liver fibrosis, in a further embodiment from advanced liver fibrosis.

Embodiment 20: The method of any one of embodiments 1 to 17, wherein said references are references for each biomarker derived from at least one subject known to not suffer from chronic liver disease, in an embodiment from liver fibrosis, in a further embodiment from advanced liver fibrosis.

Embodiment 21: The method of embodiment 20, wherein amounts for each of the biomarkers being essentially identical or similar to the corresponding references are indicative for a subject not suffering from chronic liver disease, in an embodiment from liver fibrosis, in a further embodiment from advanced liver fibrosis; and/or amounts for each of the biomarkers being different from the corresponding references are indicative for a subject suffering from chronic liver disease, in an embodiment from liver fibrosis, in a further embodiment from advanced liver fibrosis.

Embodiment 22: The method of any one of embodiment 20 or 21, wherein a decrease of IGFBP3 and/or a an increase of GGT compared to said reference is/are indicative for a subject suffering from chronic liver disease, in an embodiment from liver fibrosis, in a further embodiment from advanced liver fibrosis.

Embodiment 23: The method of any one of embodiments 1 to 22, wherein said method comprises determining at least one further biomarker, in an embodiment determining aspartate aminotransferase, alanine aminotransferase, platelet count, haptoglobin, alpha2-macroglobulin, apolipoprotein Al, bilirubin, cholesterol, hyaluronan, prothrombin index, hepatocyte growth factor (HGF), Tissue inhibitor of metalloproteinases (TIMP), and/or urea.

Embodiment 24: The method of any one of embodiments 1 to 23, wherein said method comprises further diagnostic steps, in an embodiment sonography, magnetic resonance imaging, radiography, transient elastography, and/or determining subject age and/or gender. Embodiment 25: A method for assessing chronic liver disease in a subject, said method comprising

(A) obtaining an amount of the biomarker Insulin-like growth factor-binding protein 3 (IGFBP3) in a sample from said subject;
(B) obtaining an amount of the biomarker gamma-glutamyltransferase (GGT) in said sample;
(C) comparing the amounts of the biomarkers determined in steps (a) and (b) to references for said biomarkers and/or calculating a score for assessing chronic liver disease; and
(D) assessing chronic liver disease in said subject based on the comparison and/or the calculation made in step (c).

Embodiment 26: The method of embodiment 25, wherein said method is computer-implemented.

Embodiment 27: The method of embodiment 25 or 26, wherein said method further comprises determining or having determined the amounts of said biomarker(s) in step (a) and/or (b).

Embodiment 28: The method of any one of embodiments 25 to 27 further comprising at least one feature of any one of embodiments 1 to 24.Embodiment 29: A database comprising stored references for the biomarkers IGFBP3 and GGT for assessing chronic liver disease.

Embodiment 30: The database of embodiment 29, wherein said references are references as specified in any one of embodiments 18 to 22.

Embodiment 31: The database of embodiment 29 or 30, wherein said database is tangibly embedded on a storage means.

Embodiment 32: A device comprising
(a) at least one measuring unit for determining an amount of a first biomarker being IGFBP3 and an amount of a second biomarker being GGT in a sample of the subject, said at least one measuring unit comprising at least one detection means for the first biomarker and the second biomarker; and
(b) an evaluation unit operably linked to the measuring unit, said evaluation unit comprising a data processor comprising instructions for carrying out a comparison of the amount of the first biomarker and the second biomarker to references and/or for carrying out a calculation of a score for assessing based on the amounts of the biomarkers.

Embodiment 33: The device of embodiment 32, wherein said detection means is capable of specifically detecting at least one, in an embodiment both, of said biomarkers.

Embodiment 34: The device of embodiment 32 or 33, wherein said device further comprises a database according to any one of embodiments 29 to 31 operably coupled to the data processor.

Embodiment 35: The device of any one of embodiments 32 to 34, wherein said device is a device for assessing chronic liver disease in a subject, and wherein said evaluation unit further comprises means for assessing said subject based on the comparison.

Embodiment 36: The device of any one of embodiments 32 to 35, wherein said evaluation unit is capable of automatically receiving values for the amounts of the biomarkers from the measuring unit.

Embodiment 37: The device of any one of embodiments 32 to 36, adapted to perform a method according to any one of embodiments 1 to 28, in an embodiment comprising tangibly embedded instructions which, when carried out by the data processor, cause the device to perform a method according to any one of embodiments 1 to 28.

Embodiment 38: A kit for assessing chronic liver disease in a subject comprising a first detection agent for determining an amount of IGFBP3 and second detection agent for determining an amount of GGT.

Embodiment 39: A method for assessing and treating chronic liver disease, said method comprising the steps of the method according to any one of embodiments 1 to 28 and the further step of treating said chronic liver disease in a subject identified to suffer therefrom.

Embodiment 40: Use of (i) a first biomarker being IGFBP3 and a second biomarker being GGT; and/or (ii) a first detection agent for determining an amount of IGFBP3 and a second detection agent for determining an amount of GGT, for assessing chronic liver disease.

Embodiment 41: The use of embodiment 40, comprising
(a) determining an amount of the biomarker Insulin-like growth factor-binding protein 3 (IGFBP3) in a sample from said subject;
(b) determining an amount of the biomarker gamma-glutamyltransferase (GGT) in said sample;
(c) comparing the amounts of the biomarkers determined in steps (a) and (b) to references for said biomarkers and/or calculating a score for assessing chronic liver disease; and
(d) assessing chronic liver disease in said subject based on the comparison and/or the calculation made in step (c).

Embodiment 42: The use of embodiment 40, comprising
(A) obtaining an amount of the biomarker Insulin-like growth factor-binding protein 3(IGFBP3) in a sample from said subject;
(B) obtaining an amount of the biomarker gamma-glutamyltransferase (GGT) in said sample;
(C) comparing the amounts of the biomarkers determined in steps (a) and (b) to references for said biomarkers and/or calculating a score for assessing chronic liver disease; and
(D) assessing chronic liver disease in said subject based on the comparison and/or the calculation made in step (c).

Embodiment 43: The use of any one of embodiments 40 to 42, further comprising a feature of any one of embodiments 1 to 39.

Embodiment 44: Use of a first detection agent for determining an amount of IGFBP3 and a second detection agent for determining an amount of GGT for the manufacture of a diagnostic for assessing chronic liver disease.

Embodiment 45: The use of embodiment 44, comprising
(a) determining an amount of the biomarker Insulin-like growth factor-binding protein 3 (IGFBP3) in a sample from said subject;
(b) determining an amount of the biomarker gamma-glutamyltransferase (GGT) in said sample;
(c) comparing the amounts of the biomarkers determined in steps (a) and (b) to references for said biomarkers and/or calculating a score for assessing chronic liver disease; and (

d) assessing chronic liver disease in said subject based on the comparison and/or the calculation made in step (c).

Embodiment 46: The use of embodiment 44, comprising
(A) obtaining an amount of the biomarker Insulin-like growth factor-binding protein 3 (IGFBP3) in a sample from said subject;
(B) obtaining an amount of the biomarker gamma-glutamyltransferase (GGT) in said sample;
(C) comparing the amounts of the biomarkers determined in steps (a) and (b) to references for said biomarkers and/or calculating a score for assessing chronic liver disease; and
(D) assessing chronic liver disease in said subject based on the comparison and/or the calculation made in step (c).

Embodiment 47: The use of any one of embodiments 44 to 46, further comprising a feature of any one of embodiments 1 to 39.

All references cited in this specification are herewith incorporated by reference with respect to their entire disclosure content and the disclosure content specifically mentioned in this specification.

### FIGURE LEGENDS

FIG. 1: Changes (log transformed) of individual biomarkers concentrations in in non-advanced (0) and advanced fibrosis (1). A: IGFBP3 concentration, B: GGT activity, C: IL-8 concentration. 0: controls, 1: cases.

FIG. 2. Boxplot (A) and AUROC curve (B) for IGFBP3 as a single biomarker score calculated using logistic regression model described below in Example 1. Of note the score is designed such that it is between 0 and 1 and such that it increases with decreasing IGFBP3 concentration in the sample. 0: non-advanced fibrosis, 1: advanced fibrosis.

FIG. 3. Boxplot (A) and AUROC curve (B) for IGFBP3 in combination with GGT, scores calculated using logistic regression model described below in Example 1. 0: non-advanced fibrosis, 1: advanced fibrosis.

FIG. 4. Boxplot (A) and AUROC curve (B) for IGFBP3 in combination with GGT and IL-8, scores calculated using logistic regression model described below in Example 1. 0: non-advanced fibrosis, 1: advanced fibrosis.

FIG. 5. Cross validation results for 1 (A), 2 (B), or 3 (C) biomarkers. Black rectangles indicate selected variables. The right y-axis shows the selection frequency.

FIG. 6. Boxplot (A) and AUROC curve (B) for GGT values with dataset of Example 2.

FIG. 7. Boxplot (A) and AUROC curve (B) for IGFBP3 concentrations with dataset of Example 2.

FIG. 8. Boxplot (A) and AUROC curve (B) for IGFBP3+GGT with score model trained on Example 1 dataset on Example 2 dataset.

FIG. 9. Boxplot (A) and AUROC curve (B) for IGFBP3+GGT with score model trained on Example 2 dataset on Example 2 dataset.

FIG. 10. Boxplot (A) and AUROC curve (B) for ELF assay with dataset of Example 2.

The following Examples shall merely illustrate the invention. They shall not be construed, whatsoever, to limit the scope of the invention.

### Example 1: IGFBP3 and GGT as Biomarkers of Chronic Liver Disease

The biomarkers Insulin-like growth factor-binding protein 3 (IGFBP3, Uniprot P17936), γ-glutamyltransferase (GGT, activity assay) and Interleukin-8 (IL-8, Uniprot P10145) were assessed as markers for chronic liver disease in sample panels composed of:
284 serum samples from patients with non-advanced liver fibrosis, corresponding to METAVIR score stages F0 to F2
330 serum samples from patients with advanced liver fibrosis, corresponding to METAVIR score stages F3, F4 and cirrhosis
Etiologies: HBV (majority), HCV, NASH, ASH, with approx. 66% HBV, 15% HCV and approx. 19% other etiologies.

IGFBP3 and GGT were measured with Roche IVD assays. For measurement of IL-8 a Roche prototype platform IMPACT was used (Chandra et al., Arthritis Research & Therapy 2011, 13 :R102). AUCROC was calculated for differentiation of non-advanced from advanced fibrosis for the individual biomarkers and the combination of biomarkers and increased with the number of combined biomarkers.

Scores based on single biomarkers or biomarker combinations were calculated as follows: the amounts of the biomarker(s) determined were log10 transformed and linearly combined with corresponding coefficient(s) (c₁, . . . , Cₙ for n biomarkers, wherein n is the number of biomarkers) and intercept (c₀). The resulting linear combination (x) was then used as input to a sigmoid transformation (f(x)=1/(1+exp(−x))) providing a score, which maps the output range to 0-1 and which is proportional to the risk for aggressive liver fibrosis. An exhaustive search in a nested cross-validation scheme, with the biomarker data split randomly into training (3/4) and test (1/4) sets was performed to determine the optimal coefficient(s) and intercept for the model to calculate the score and assess the clinical performance. The optimal model for calculating the score was selected (i.e. in particular the coefficients and intercepts were optimized) using an exhaustive search (i.e. all possible biomarker combinations were assessed) with an inner cross-validation of the training set and tested on the remaining test data set. This procedure was repeated a hundred times. The test data in the outer loop was used to evaluate the clinical performance of the optimal model for calculating the score by determining the AUROC.

### Example 1.1: IGFBP3, GGT, and IL-8 as Single Markers

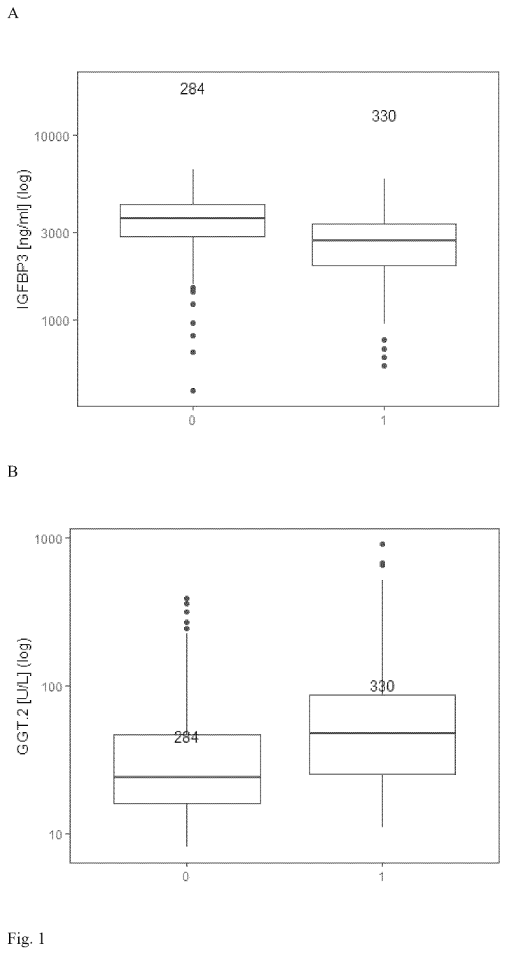

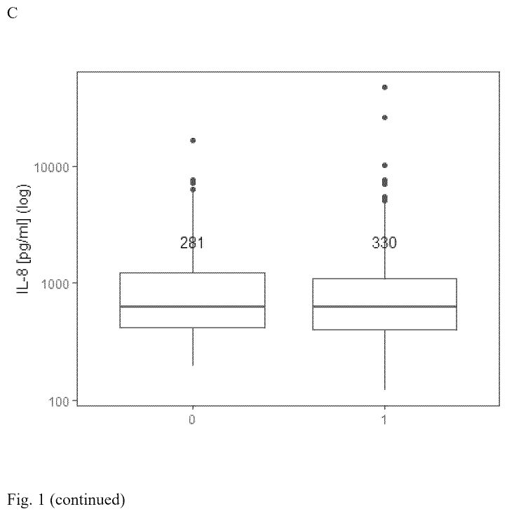

Changes of the single markers of the panel are shown in FIG. 1.

IGFBP3 concentrations in serum/plasma of liver fibrosis patients significantly decrease in advanced (F3, F4, cirrhosis) disease compared to non-advanced fibrosis (F0, F1 and F2).

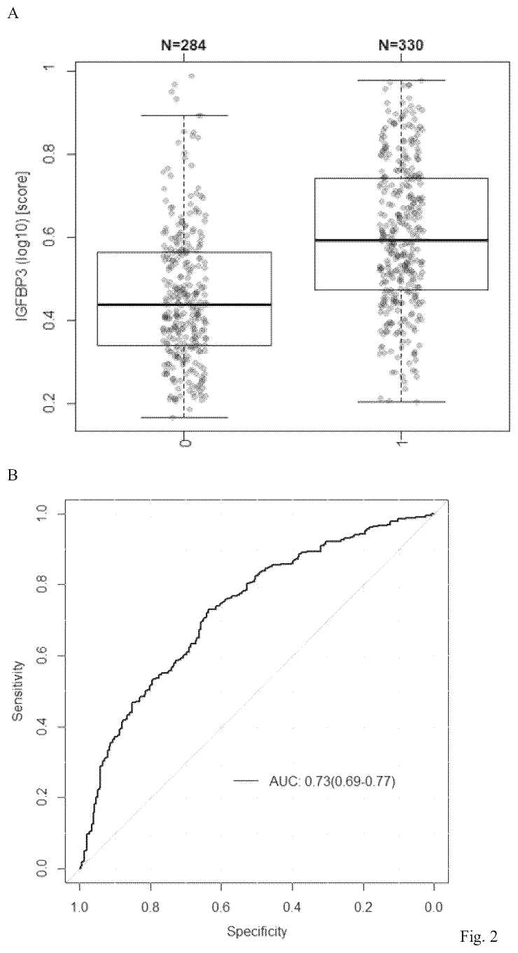

AUROC of IGFBP3 as a single marker using a single biomarker score as described in Example 1 above for differentiation between non-advanced and advanced fibrosis in the measured cohort was 0.73 (0.69-0.77) (FIG. 2). Exemplary cutoff values for the score based on IGFBP3 as single biomarker with resulting specificity and sensitivity values are shown in Table 1 below.

TABLE 1
Exemplary cutoff values with resulting specificity and
sensitivity values for IGFBP3 as single marker score.

| | Cutoff | | | | |
|---|---|---|---|---|---|
| Cutoff Criterion | [score] | Sensitivity (95%-CI) | | Specificity (95%-CI) | |
| Spec = 0.95 | 0.77 | 0.2 | (0.16-0.25) | 0.95 | (0.92-0.97) |
| Spec = 0.9 | 0.66 | 0.37 | (0.32-0.42) | 0.9 | (0.86-0.93) |
| Sens = 0.95 | 0.32 | 0.95 | (0.92-0.97) | 0.19 | (0.15-0.24) |
| Sens = 0.9 | 0.38 | 0.9 | (0.86-0.93) | 0.32 | (0.27-0.38) |

AUROC of GGT as a single marker for differentiation between non-advanced and advanced fibrosis in measured cohort was 0.68 (0.64-0.72); IL-8 performance as a single biomarker for differentiation of advanced from non-advanced fibrosis was rather weak with AUROC 0.51 (0.47-0.56).

### Example 1.2: Two Biomarker Panel: IGFBP3+GGT

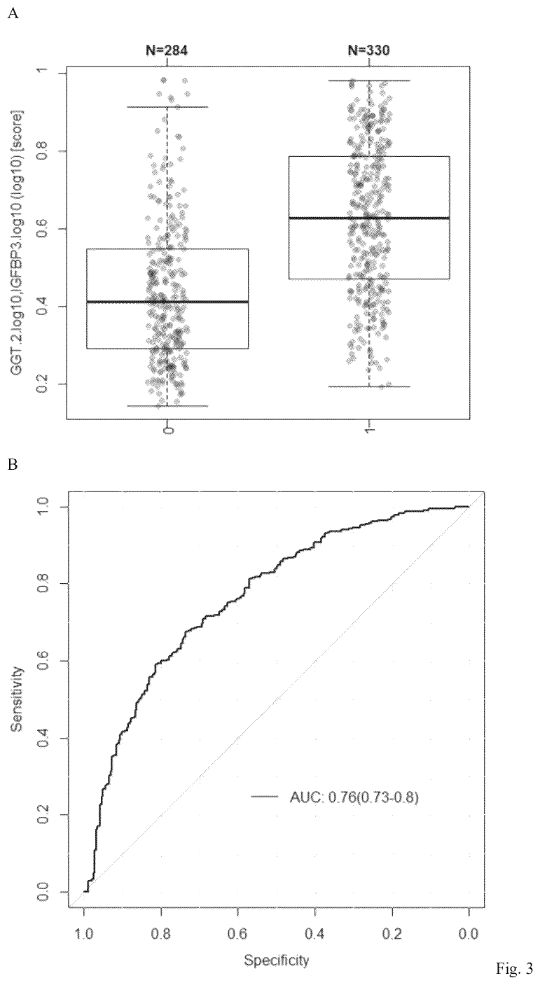

Performance of the two biomarker panel: IGFBP3+GGT (combined in a score as described in Example 1) is summarized in FIG. 3. Exemplary cutoff values with resulting specificity and sensitivity values are shown in Table 2 below. AUROC of GGT in combination with IGFBP3 was 0.76 (0.72-0.79) (FIG. 3). Exemplary cutoff values with resulting specificity and sensitivity values are shown in Table 2 below.

TABLE 2
Exemplary cutoff values with resulting specificity and sensitivity
values for IGFBP3 + GGT as two-marker panel score.

| | Cutoff | | | | |
|---|---|---|---|---|---|
| Cutoff Criterion | [score] | Sensitivity (95%-CI) | | Specificity (95%-CI) | |
| Spec = 0.95 | 0.78 | 0.27 | (0.22-0.32) | 0.95 | (0.92-0.97) |
| Spec = 0.9 | 0.68 | 0.42 | (0.36-0.47) | 0.9 | (0.86-0.93) |
| Sens = 0.95 | 0.31 | 0.95 | (0.92-0.97) | 0.29 | (0.24-0.34) |
| Sens = 0.9 | 0.36 | 0.9 | (0.86-0.93) | 0.4 | (0.35-0.46) |

### Example 1.3: Three-Biomarker Panel: IGFBP3+GGT+IL-8

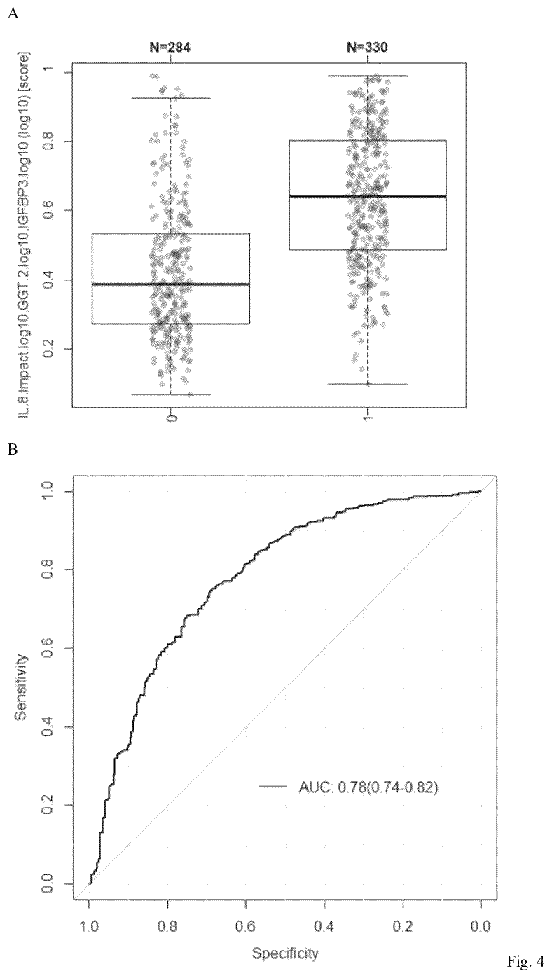

Performance of the three biomarker panel: IGFBP3+GGT+IL-8 (combined in a model calculating a score as described in Example 1) is summarized in FIG. 4. Inclusion of IL-8 increased the AUROC of IGFBP3 and GGT combination to 0.78 (0.74-0.82). Exemplary cutoff values with resulting specificity and sensitivity values are shown in Table 3 below.

TABLE 3
Exemplary cutoff values with resulting specificity and sensitivity
values for IGFBP3 in combination with GGT and IL-8 as score.

| | Cutoff | | | | |
|---|---|---|---|---|---|
| Cutoff Criterion | [score] | Sensitivity (95%-CI) | | Specificity (95%-CI) | |
| Spec = 0.95 | 0.82 | 0.22 | (0.18-0.27) | 0.95 | (0.92-0.97) |
| Spec = 0.9 | 0.73 | 0.35 | (0.3-0.4) | 0.9 | (0.86-0.93) |
| Sens = 0.95 | 0.31 | 0.95 | (0.92-0.97) | 0.36 | (0.3-0.41) |
| Sens = 0.9 | 0.38 | 0.9 | (0.86-0.93) | 0.48 | (0.42-0.54) |

### Example 1.4: Comparison of the Biomarker Panels of Examples 1.2 and 1.3 to Known Biomarkers and Combinations Thereof

The biomarker panels of Examples 1.2 and 1.3 were compared to known biomarkers of chronic liver disease and combinations of two or three of these biomarkers; the biomarkers evaluated were
IL-10, IL-8, BMP7, COMP, DKK1, OPG, ALB, GGT, BILT, AFU, GLDH, ALT, AST, GP73, HGF, TIMP-1, AXL, Midkine, CES1, Ang-2, OPN, GPC3, MMP3, CEA, Cyfra 21-1, Ferritin, HE4, IL-6, NSE, CA 125, CA 15.3, CA 19-9, CA72.4, IGF1, IGFBP3, IGFBP7, MMP2,Sialyltransferase, AFP-L3, AFP, PIVKA-II, PRO-C3, PRO-C3X, Seprase, age, and gender.

To evaluate the performance of the biomarkers and biomarker panels of Examples 1.1 to 1.3 a cross-validation using Uni-and Multivariate analyses were performed. This allows to assess whether the biomarkers or biomarker combinations of Examples 1.1 to 1.3 provide a better differentiation of non-advanced from advanced liver fibrosis compared to any of the other tested biomarkers or any combinations thereof.

In detail, estimation of diagnostic accuracy of biomarker combinations was done by an exhaustive search in a nested cross-validation scheme, with the biomarker data split randomly into training (3/4) and test (1/4) sets. The optimal model was selected (i.e. in particular the coefficients and intercepts were optimized) using an exhaustive search (i.e. all possible biomarker combinations were assessed) with an inner cross-validation of the training set and tested on the remaining test data set. This procedure was repeated a hundred times. The test data in the outer loop was used to evaluate the clinical performance of the optimal model. The nested cross-validation procedure provided the selection frequency of the best marker combinations and a robust estimate of the performance (AUCROC) for each model (to avoid overfitting).

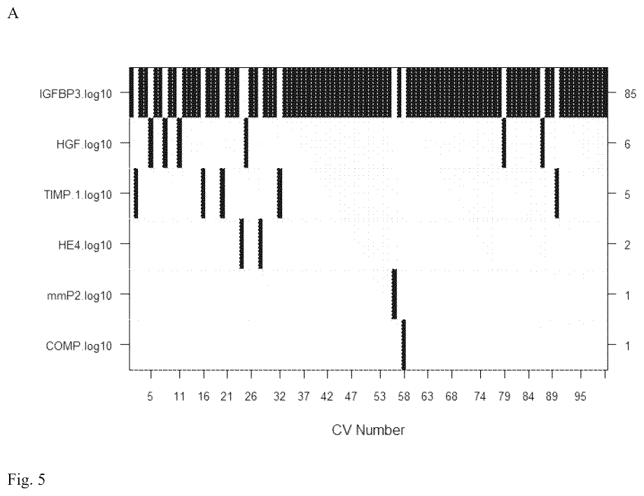

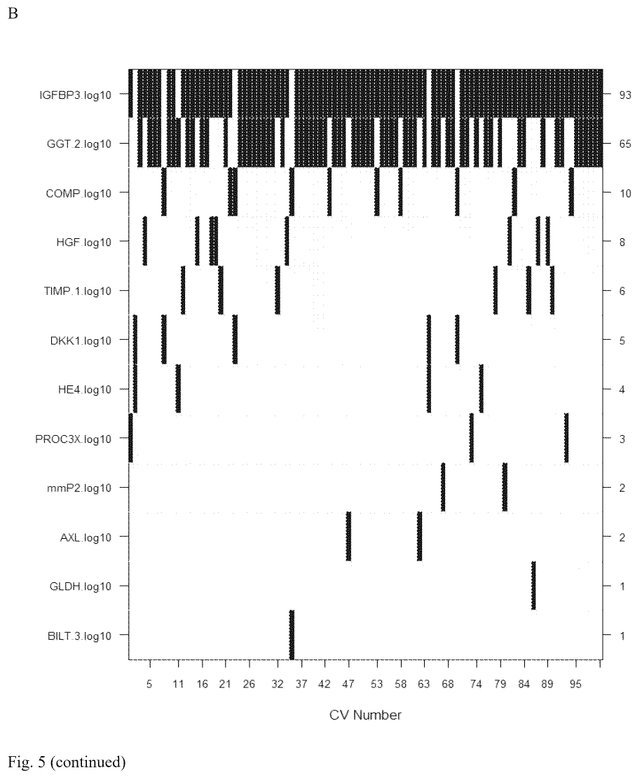

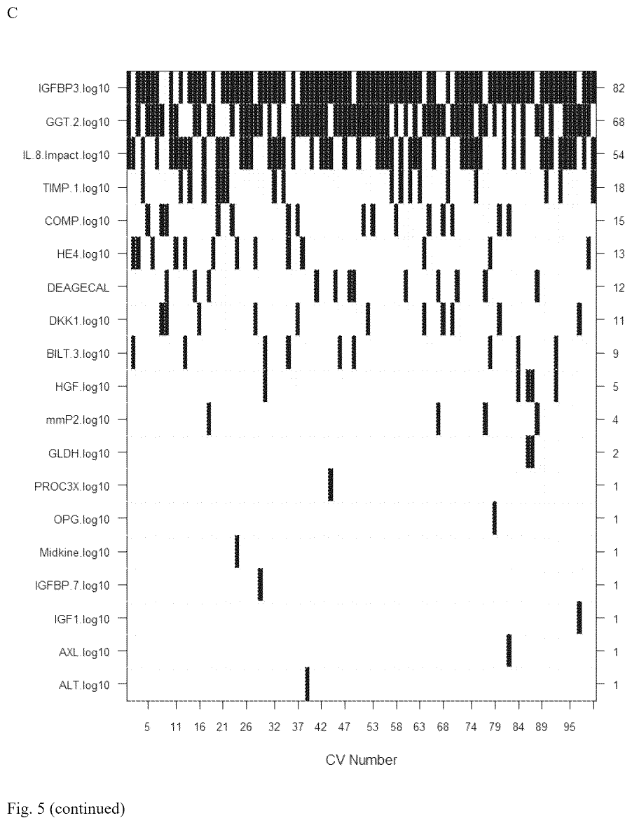

IGFBP3 was the most frequently selected feature in one, two and three biomarker combinations, with the 85%, 93% and 82% frequency, respectively. GGT was selected as a second feature with 65% and 68% frequency. IL8 was selected as a third feature with 54% frequency (FIG. 5A)). Accordingly, this cross validation clearly indicates that IGFBP3 is the best individual biomarker for differentiation of non-advanced from advanced liver fibrosis in the studied cohort. In addition, this cross validation surprisingly demonstrates that the multivariate combination of GGT among the huge variety of other biomarkers improves differentiation of non-advanced from advanced liver fibrosis the best among all tested two biomarker combinations (FIG. 5 B)). Of note, many two biomarker combinations did not at all improve performance in differentiation of non-advanced from advanced liver fibrosis vis-à-vis the single biomarkers. It is a novel and surprising finding that a combination of IGFBP3 and GGT provides for such superior differentiation of non-advanced from advanced liver fibrosis compared to all other two biomarker combinations. Another surprising finding is that adding IL-8 as third biomarker into the multivariate analyses even further improves differentiation of non-advanced from advanced liver fibrosis and seems superior to any other three biomarker combination of the tested biomarkers (FIG. 5c)).

All in all, this cross validation analyses demonstrate that the combination of IGFBP3 and GGT (and optionally IL-8) provides a superior performance in differentiation of non-advanced from advanced liver fibrosis compared to any other combinations of the other tested biomarkers, which include well known markers of chronic liver disease.

Mean AUROC for the combination of IGFBP3, GGT and optionally IL8 for differentiation between non-advanced (F0, F1 and F2) and advanced (F3, F4) fibrosis+cirrhosis are described above in Examples 1.1 to 1.3.

### Example 2: Evaluation of Biomarkers in NASH Cohort

Clinical performance of the biomarkers and clinical data: Siemens ELF score: HA, TIMP-1, PIIINP; GGT and IGFBP3 were evaluated in sample panel composed of:
125 serum samples from patients with confirmed NASH fibrosis,
82 serum samples from controls including NAFLD (n=17) and apparently healthy donors.

Biomarkers and biomarker panel were evaluated for differentiation of control panel (NAFLD+healthy) from NASH liver fibrosis. IGFBP3, GGT, were measured with Roche IVD or Robust Prototype Elecsys assays. ELF panel was measured on Siemens Atellica analyzer.

### Example 2.1 GGT and IGFBP3 as Single Biomarkers

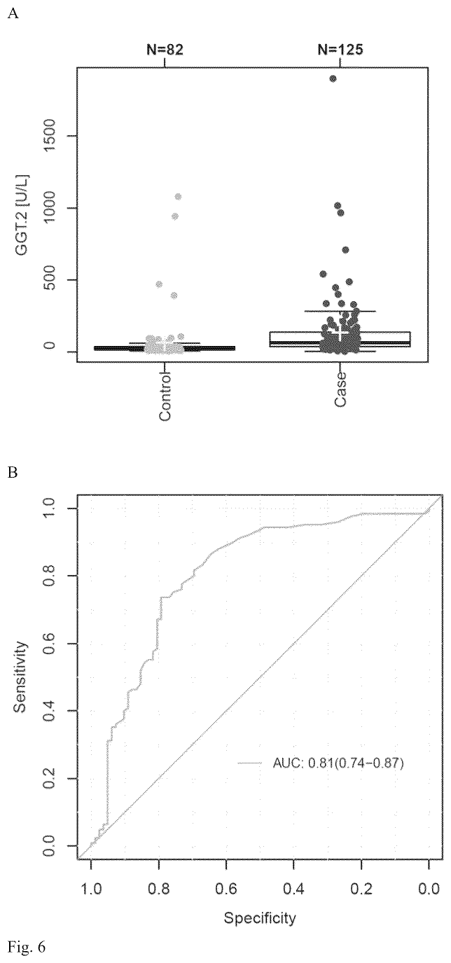

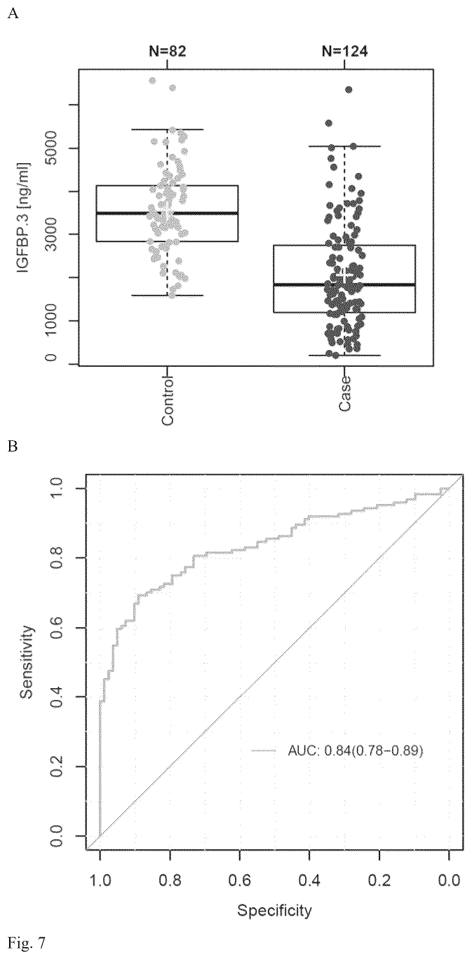

Results for GGT and IGFBP3 are summarized in FIGS. 6 and 7, respectively. Exemplary cutoff values with resulting specificity and sensitivity values are shown in Tables 4 and 5 below.

TABLE 4
Exemplary cutoff values with resulting specificity
and sensitivity values for GGT as single marker.

| | Cutoff | | | | |
|---|---|---|---|---|---|
| Cutoff Criterion | [U/L] | Sensitivity (95%-CI) | | Specificity (95%-CI) | |
| Spec = 0.95 | 109 | 0.31 | (0.24-0.4) | 0.95 | (0.88-0.98) |
| Spec = 0.9 | 90 | 0.4 | (0.32-0.49) | 0.9 | (0.82-0.95) |
| Sens = 0.95 | 18 | 0.95 | (0.9-0.98) | 0.37 | (0.3-0.47) |
| Sens = 0.9 | 24 | 0.9 | (0.83-0.94) | 0.59 | (0.48-0.69) |

TABLE 5
Exemplary cutoff values with resulting specificity
and sensitivity values for IGFBP3 as single marker

| | Cutoff | | | | |
|---|---|---|---|---|---|
| Cutoff Criterion | [ng/ml] | Sensitivity (95%-CI) | | Specificity (95%-CI) | |
| Spec = 0.95 | 2029 | 0.6 | (0.51-0.68) | 0.95 | (0.88-0.98) |
| Spec = 0.9 | 2265 | 0.67 | (0.58-0.75) | 0.9 | (0.82-0.95) |
| Sens = 0.95 | 4357 | 0.95 | (0.9-0.98) | 0.21 | (0.13-0.31) |
| Sens = 0.9 | 3716 | 0.9 | (0.84-0.94) | 0.41 | (0.31-0.52) |

### Example 2.2: Two Biomarker Panel: IGFBP3+GGT

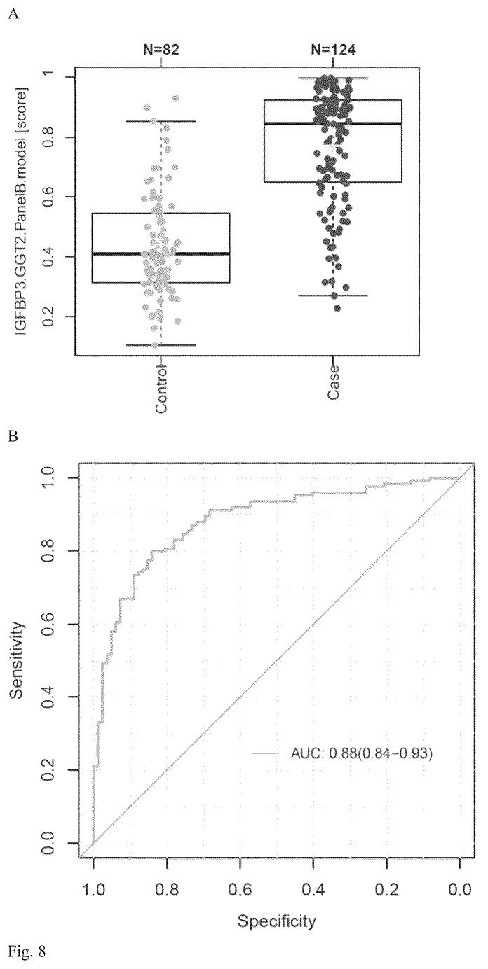

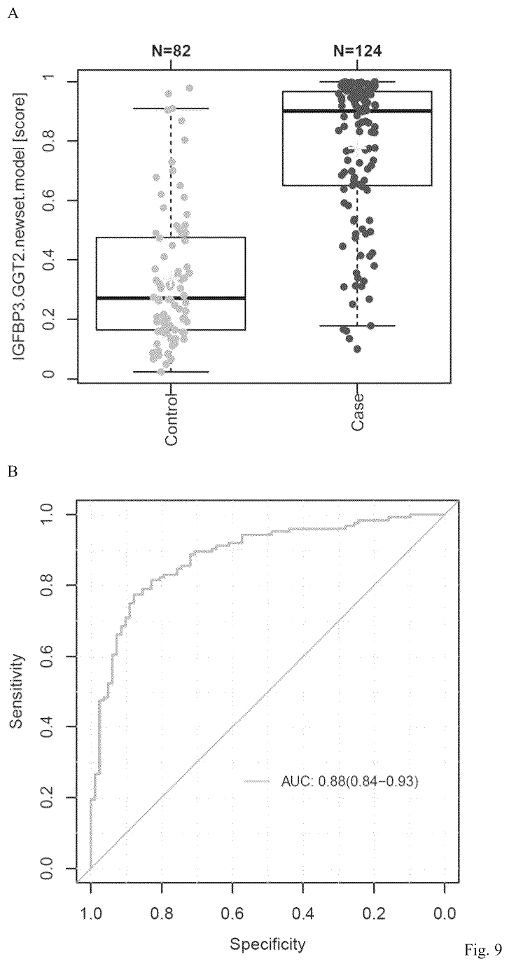

Performances of the two biomarker panel: IGFBP3+GGT on the data of Example 2, are summarized in FIGS. 8 and 9. Exemplary cutoff values with resulting specificity and sensitivity values are shown in Tables 6 and 7 below. Performance was calculated (i) using a score model trained on Example 1 dataset on Example 2 dataset (FIG. 8) and (ii) using a score model trained on Example 2 dataset on Example 2 dataset (FIG. 9), respectively. Both models resulted in same performance, which demonstrates robustness of the biomarker panel and its applicability to different liver fibrosis etiologies.

TABLE 6
Exemplary cutoff values with resulting specificity and sensitivity
values for IGFBP3 + GGT as marker panel score; data of
Example 2 samples using model trained on Example 1 data set.

| | Cutoff | | | | |
|---|---|---|---|---|---|
| Cutoff Criterion | [score] | Sensitivity (95%-CI) | | Specificity (95%-CI) | |
| Spec = 0.95 | 0.79 | 0.58 | (0.49-0.66) | 0.95 | (0.88-0.98) |
| Spec = 0.9 | 0.7 | 0.67 | (0.58-0.75) | 0.9 | (0.82-0.95) |
| Sens = 0.95 | 0.39 | 0.95 | (0.9-0.98) | 0.45 | (0.35-0.77) |
| Sens = 0.9 | 0.48 | 0.9 | (0.84-0.94) | 0.68 | (0.58-0.77) |

TABLE 7
Exemplary cutoff values with resulting specificity and sensitivity
values for IGFBP3 + GGT as marker panel score; data of
Example 2 samples using model trained on Example 2 data set.

| | Cutoff | | | | |
|---|---|---|---|---|---|
| Cutoff Criterion | [score] | Sensitivity (95%-CI) | | Specificity (95%-CI) | |
| Spec = 0.95 | 0.88 | 0.52 | (0.44-0.61) | 0.95 | (0.88-0.98) |
| Spec = 0.9 | 0.68 | 0.71 | (0.62-0.78) | 0.9 | (0.82-0.95) |
| Sens = 0.95 | 0.27 | 0.95 | (0.9-0.98) | 0.49 | (0.38-0.59) |
| Sens = 0.9 | 0.35 | 0.9 | (0.84-0.94) | 0.68 | (0.55-0.75) |

### Example 2.3: Comparative Example: ELF test

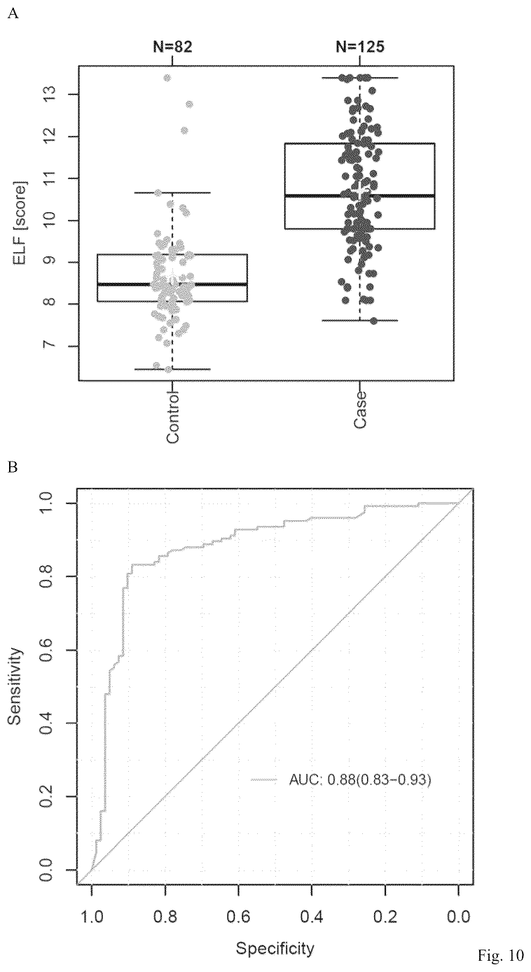

For comparison, performance of the known biomarker panel of the ELF test (Siemens Healthineers International AG) was evaluated on the same cohorts as described in Example 2. This test is a widely used blood test for fibrosis, comprising determination of hyaluronic acid (HA), aminoterminal propetide of procollagen type III (PIIINP), and tissue inhibitor of metalloproteinase 1 (TIMP-1). The results obtained are shown in FIG. 10; Exemplary cutoff values with resulting specificity and sensitivity values are shown in Table 8 below.

TABLE 8
Exemplary cutoff values with resulting specificity
and sensitivity values for the ELF marker panel.

| | Cutoff | | | | |
|---|---|---|---|---|---|
| Cutoff Criterion | [score] | Sensitivity (95%-CI) | | Specificity (95%-CI) | |
| Spec = 0.95 | 10 | 0.54 | (0.46-0.63) | 0.95 | (0.88-0.98) |
| Spec = 0.9 | 9.6 | 0.81 | (0.73-0.87) | 0.9 | (0.82-0.95) |
| Sens = 0.95 | 8.4 | 0.95 | (0.9-0.98) | 0.48 | (0.37-0.58) |
| Sens = 0.9 | 9 | 0.9 | (0.84-0.94) | 0.67 | (0.56-0.76) |

### Example 2.4: Conclusion

As is clear from Example 2, the marker panel proposed herein is superior to known biomarker panels in differentiation of NASH fibrosis from NAFLD and healthy cases, or at least comparable. Importantly, IGFBP3+GGT combination performance was identical independently of the model training data used for the data evaluation. We have evaluated this data set first using the model for combining IGFBP3 and GGT as trained on Example 1 data, based mainly on viral hepatitis etiologies, followed by independent evaluation using model trained on Example 2 data, based exclusively on non-viral hepatitis. As demonstrated in FIGS. 8 and 9, AUCROC for both evaluations are identical, thus demonstrating an universal utility of the IGFBP3+GGT biomarker combination for assessment of liver fibrosis independent of the etiology. It is of particular note that the panel of IGFBP3 and GGT comprises only two biomarkers instead of three biomarkers as used in ELF, which is the most popular biomarker solution in the field. This is a particular advantage as it involves fewer resources and is easier. Taking into account the data of Example 1.3 it also appears plausible that a panel combining IGFBP3, GGT and IL-8 also shows a better differentiation of control panel (NAFLD+healthy) from NASH liver fibrosis than ELF.

### LITERATURE

Bataller & Brenner (2005), J Clin Invest 115:209
Castera et al., Hepatology 2010;51:828-835
Chowdhury and Mehta (2022), Clin Exp Med, doi.org/10.1007/s10238-022-00799-z
Claudon et al. (2008), Clinical Chemistry 54(9): 1463
Correa et al., World J Hepatol 2016; 8(17): 739-748
Dillon et al., Annals of Clinical Biochemistry 2016, Vol. 53(6) 629-631
Estes et al. (2018), J Hepatol.69(4):896
EASL-EASD-EASO Clinical Practice Guidelines for the management of non-alcoholic fatty liver disease. J Hepatol 2016; 64:1388-1402
Glass et al., Hepatol Commun. 2018;2:1344-1355
Guo & Lu (2020), J Clin transl Hepatol 8(3):304
Karsdal et al., Liver Int 2020; 40:736-750
Lurie et al., World J Gastroenterol 2015; 21(41): 11567-11583
Ophir et al., Ultrasound Imaging 1991; 13:111-134
Tsukada et al, Clin Chim Acta 2006; 364:33-60
Volzke et al., European Journal of Endocrinology 2009; 161(5): 705-713
Xie et al, PLOS One 2014, doi.org/10.1371/journal.pone.0092772
Zweig 1993, Clin. Chem. 39:561-577

1. A method for assessing chronic liver disease in a subject, said method comprising
(a) determining an amount of the biomarker Insulin-like growth factor-binding protein 3 (IGFBP3) in a sample from said subject;
(b) determining an amount of the biomarker gamma-glutamyltransferase (GGT) in said sample;
(c) comparing the amounts of the biomarkers determined in steps (a) and (b) to references for said biomarkers and/or calculating a score for assessing chronic liver disease; and
(d) assessing chronic liver disease in said subject based on the comparison and/or the calculation made in step (c).

2. The method of claim 1, wherein said chronic liver disease is liver fibrosis.

3. The method of claim 1, wherein said assessing comprises diagnosing liver fibrosis, comprises staging chronic liver disease, comprises staging liver fibrosis, comprises differentiating between non-advanced liver fibrosis and advanced liver fibrosis, and/or comprises excluding advanced liver fibrosis.

4. The method of claim 1, wherein said method is comprised in a method of monitoring chronic liver disease.

5. The method of claim 1, wherein said method further comprises the steps of determining an amount of the biomarker Interleukin-8 (IL-8), and wherein step (c) comprises comparing the amounts of the three biomarkers determined to references for said biomarkers and/or calculating a score for assessing chronic liver disease.

6. The method of claim 1 wherein the amount of the biomarker IGFBP3 and optionally the amount of the biomarker IL-8 is/are determined by an immunoassay.

7. The method of claim 1, wherein the amount of the biomarker GGT is determined by an activity assay.

8. The method of claim 1, wherein said sample is a bodily fluid sample.

9. The method of claim 1, wherein said method comprises determining at least one further biomarker.

10. The method of claim 1, wherein said method comprises further diagnostic steps.

11. A computer-implemented method for assessing chronic liver disease in a subject, said method comprising
(A) obtaining an amount of the biomarker Insulin-like growth factor-binding protein 3 (IGFBP3) in a sample from said subject;
(B) obtaining an amount of the biomarker gamma-glutamyltransferase (GGT) in said sample;
(C) comparing the amounts of the biomarkers determined in steps (a) and (b) to references for said biomarkers and/or calculating a score for assessing chronic liver disease; and
(D) assessing chronic liver disease in said subject based on the comparison and/or the calculation made in step (c).

12. (canceled)

13. (canceled)

14. A device comprising
(a) at least one measuring unit for determining an amount of a first biomarker being IGFBP3 and an amount of a second biomarker being GGT in a sample of the subject, said at least one measuring unit comprising at least one detection means for the first biomarker and the second biomarker; and
(b) an evaluation unit operably linked to the measuring unit, said evaluation unit comprising a data processor comprising instructions for carrying out a comparison of the amount of the first biomarker and the second biomarker to references and/or for carrying out a calculation of a score for assessing based on the amounts of the biomarkers.

15. (canceled)

16. The method of claim 2, wherein said chronic liver disease is selected from advanced liver fibrosis, advanced liver fibrosis corresponding to METAVIR score stage F3 or F4, and/or cirrhosis.

17. The method of claim 8, wherein said body fluid sample is selected from a blood sample, a plasma sample, and/or a serum sample.

18. The method of claim 9, wherein the at least one further biomarker is selected from aspartate aminotransferase, alanine aminotransferase, platelet count, haptoglobin, alpha2-macroglobulin, apolipoprotein A1, bilirubin, cholesterol, hyaluronan, prothrombin index, hepatocyte growth factor (HGF), Tissue inhibitor of metalloproteinases (TIMP), and/or urea.

19. The method of claim 10, wherein said further diagnostic steps are selected from sonography, magnetic resonance imaging, radiography, transient elastography, and/or determining subject age and/or gender.

20. A method of detecting a decrease in the amount of the biomarker Insulin-like growth factor-binding protein 3 (IGFBP3) of claim 1 and an increase in the amount of the biomarker gamma-glutamyltransferase (GGT) of claim 1 in a subject, the method comprising:
measuring an amount of IGFBP3 in a sample from said subject;
measuring an amount of GGT in said sample; and
detecting the decrease in the amount of IGFBP3 and the increase in the amount of GGT in the sample of the subject by contacting the sample with a detection agent for IGFBP3 and a detection agent for GGT, wherein the binding between IGFBP3 and the detection agent for IGFBP3 is detected, and wherein the binding between GGT and the detection agent for GGT is detected.

21. A method for measuring a panel of biomarkers in a subject suspected to suffer from chronic liver disease or suffering from chronic liver disease, the method comprising:
obtaining a sample from the subject;
determining a measurement for the panel of biomarkers in the sample, wherein the panel comprises the biomarkers Insulin-like growth factor-binding protein 3 (IGFBP3) and gamma-glutamyltransferase (GGT), wherein the measurement comprises determining a level of each of the biomarkers IGFBP3 and GGT in the panel.

22. The method of claim 20, further comprising:
measuring an amount of the biomarker Interleukin-8 (IL-8) in said sample; and
detecting whether IL-8 is present in said sample by contacting the sample with a detection agent for IL-8, wherein the binding between IL-8 and the detection agent for IL-8 is detected.

23. The method of claim 21, wherein the panel further comprises the biomarker Interleukin-8 (IL-8), and wherein the measurement further comprises determining a level of the biomarker IL-8 in the panel.
